(19) United States
(12) Patent Application Publication
(10) Pub. No.: US 2005/0048020 A1
(43) Pub. Date: Mar. 3, 2005

(54) Novel topical delivery system for plant derived anti-irritants

(76) Inventor: John J. Wille, Trenton, NJ (US)

Correspondence Address: Nancy A. Bird, 231 Walton Ave, South Orange, NJ, 07079, US

(21) Appl. No.: 10/873,590

(22) Filed: Jun. 22, 2004

Related U.S. Application Data
(60) Provisional application No. 60/485,058, filed on Jul. 7, 2003.

(51) Int. Cl. A61K 7/06; A61K 7/11; A61K 7/075; A61K 7/08

(52) U.S. Cl. 424/70.13; 424/70.24

(57) ABSTRACT

A thixotropic microemulsion for the treatment of mammalian skin; and methods for the preparation thereof; and skin treatment formulations formed therefrom. The delivery system is an oil-in-water type emulsion that is especially useful as a vehicle for the delivery of hydrophobic and hydroalcoholic plant extracts having anti-irritant and/or anti-oxidant properties. The delivery system is comprised of natural, hydrophobic, chemically-modified type starches and polysaccharaides. Among the oils preferred in this embodiment are: mineral oil, petrolatum jelly and silicone oils, either added singly or in various combinations. Among surfactants employed as emulsifiers are: either a cationic detergent, e.g., benzalkonium chloride, an anionic, e.g., sodium lauryl sulfate, and a non-ionic detergent, e.g., Triton X-100 or combinations of oils and surfactants. The resulting emulsion is a semi-viscous and thixotropic gel, which is stable upon prolonged standing at ambient storage temperature.

### RELATED APPLICATIONS

This application claims priority from Provisional Application 60/485,058, filed Jul. 7, 2004, entitled “Novel Topical Delivery System for Plant Derived Anti-irritants”.

### FIELD OF THE INVENTION

The field of the invention is in cosmetic and personal care formulations and in the topical delivery of anti-irritant actives derived from plant material.

### BACKGROUND OF THE INVENTION

The creation of cosmetic and personal care product formulations containing multiple ingredients presents many difficulties and challenges due to the unanticipated behavior of any particular ingredient in the final formulation. A formulation with an unstable pH leads to loss of activity of a key ingredient. For instance, lactic acid is ineffective at neutral pH, and a shift in hydrogen ion concentration of the formulation over time, toward a neutral pH, may cancel the hoped-for activity of the lactic acid. In addition, oxidation reactions in aqueous formulations may destroy the activity of key ingredients added to provide protection against ultra-violet light induced skin damage. As an example, ascorbic acid (Vitamin C) is often highly unstable in aqueous formulations due to free radical reactions that occur in the final formulation over time.

In addition to the time induced instabilities, there are basic challenges arising from the hydrophobicity of many new key ingredients, which may result in their being insoluble in oil-in-water emulsion formulations; and water-in-oil ointment formulations are generally considered too greasy for high performance cosmetics and personal care products. Earlier attempts to create new oil-in-water formulations include a jet cooking process disclosed in Eskins and Fanta. U.S. Pat. No. 5,882,713 (1999) and Eskins and Fanta. U.S. Pat. No. 5,676,994 (1997). These patents describe methods to form a stable colloidal dispersion of oil in an aqueous starch matrix. They have found applications in the food industry as thickeners and stabilizers, and in the water-proofing and sizing of paper. The jet cooking process totally solubilizes the starch grains, which are then thoroughly mixed at elevated temperatures and at excess steam jet pressures. The turbulence created by the excess steam pressure. disperses the oil droplets in the starch matrix. The method produces a stable dispersion of oil in an aqueous starch matrix without the need for an emulsifier. The stability of the dispersion has been shown to arise from the steam jet cooking process that deposits starch molecules at the oil-water interface, encapsulating micron-sized oil droplets, ( Eskins et al, 1996; Fanta and Eskins, 1998; Fanta et al, 199a; Fanta et al., 1999b). The carbohydrate-coated oil droplets are unable to coalesce upon cooling, and the solubilized starch is largely prevented from undergoing retrorade gelation. The technology was trademarked as the Fantesk™ process. In later studies these inventors reported that natural starches do contain small amounts of endogenous emulsifying agents including phospholipids and phosphoproteins, which may account for some of the emulsifying potential of high molecular weight natural starches

A license for certain medical applications of this technology was awarded to Hygene, Inc. under a Cooperative Research and Development agreement with the United States Department of Agriculture. This led to several publications reporting the utility of the technology for delivery of hydrocortisone (Wille al, 2000), as a skin protective lotion (Burdge et al, 2000) and as a vehicle for anti-microbial hand lotion (Wille et al, 2002).

However, in the personal care and cosmetic arena, natural starches have been passed over as cosmetic ingredients in favor of modified starches, due to their undesirable gel and film-forming properties.

A major consideration in developing personal care and cosmetic products is the anticipated regulatory review concerning both the safety and the efficacy of the ingredients to be incorporated into both the delivery system, and the “active ingredients” to be incorporated therein. The U.S. Food and Drug Administration (FDA) approves and regulates the sale of drugs to certified claims and indications for a drug. By contrast, the FDA restricts the sale of cosmetics to claims that affect the beauty and appearance of skin, without altering skin structure, in order to reduce skin irritation. But consumers have become more sophisticated in choosing cosmetic products in the hope of actually slowing the effects of skin aging and facial wrinkling, and cosmetic companies have sought to provide the consumers with a sound scientific rationale to underpin their product's superiority in affecting the skin appearance.

Among the active agents which have been approved for topical, cosmetic use by the FDA are: Vitamins A, E and K,( i.e., fat-soluble vitamins), alpha-hydroxy acid (AHAs, e.g., glycolic acid and lactic acid at or below 5%): anti-wrinkling and skin exfloliants; and retinoids ( i.e., Vitamin A derivatives), e.g., RetinA, (Retinoic Acid, 0.01% creams). The use of active agents has been a prime source of confusion since such materials are legally neither drugs nor cosmetics. Such materials have been termed cosmeceuticals. Other such “active” compounds approved for topical, cosmetic use include: glucans, enzymes, and antioxidants. By and large the cosmeceuticals now being marketed in the US have been “grandfathered in” by the Cosmetic Fragrance and Toiletries regulatory approval process.

The cosmetic industry is always seeking novel and high technology solutions for the delivery of “active agents”. This has led to an explosion in the cosmetic-related patent literature, with more than 400 patents issuing per year over the last decade. The majority of these concern novel and innovative cosmetic delivery systems. Although the majority of cosmetic products today are based on oil-in-water or water-in-oil emulsions, much effort has been expended improving the “feel” of formulations through 1) changes in rheological properties, 2) attempting to eliminate an “oily feel” by compositional changes, or 3) by optimizing pH and surfactant emulsifier systems by using milder emulsifying surfactants and emulsion stabilizing chemicals in order to reduce skin irritation. Some systems have been developed without surfactants at all.

However, the ideal cosmetic delivery system must do more than just deliver the “active agents” because many cosmeceutical agents are themselves skin irritants, when used at or near concentrations required to deliver true activity. It is for this reason that new natural compounds, or botanical agents, have been sought. These plant-derived anti-irritants can be added to personal care formulations to reduce the skin irritation caused by other, more common actives such as AHAs and retinoids. Similar approaches to reducing irritation include the use of Bisabolol, a known natural anti-irritant, the use of artificial mixtures of skin lipids to normalize irritated skin.

Several patents have been issued describing plant extracts that are useful as anti-irritants in cosmetic formulations. For example, Castro (1995) in U.S. Pat. No. 5,393,526 (assigned to E, Arden) discloses the use of Rosmarinic acid derived from extracts of Sage. Smith, et al (1989) in European Patent Application No. 0,3554554A2 (assigned to E. Arden), disclosed the use of plant extracts from the Cola nitida plant. Parnell (1991) in WO 9, 114,441(assigned to Parnell Pharma), discloses Yerba oil, and bis Abolol and Chamazulene as anti-irritants. Finally, Govil and Kohlman (1990) in U.S. Pat. No. 4,908,213, disclose the anti-irritant properties of the Yarrow plant extracts.

Another area of research in the personal care and cosmetic industry is for a product that can deliver topical oxygen. In this regard, the topical application of oxygen has demonstrated benefits for both wound care applications and for the improvement of the beauty and appearance of skin. A topical gel conveying oxygen to the skin via a controlled release delivery system is one of the goals of formulators for cosmetic, skin care and wound healing applications, including the development of an asymmetric oxygen carrier system (Stanzl, 1999). Once released on the skin from an appropriate vehicle, the vehicle should act to trap and slowly release the dissolved oxygen. This can be accomplished by starch films which are known to be virtually impermeable to oxygen and air. The invention described in the present application develops formulations that incorporate perfluorocarbon. Many patents disclose the use a perfluorocarbon to bind oxygen and to deliver it in a formulation, some of which are: Moore. U.S. Pat. No. 4,569,784, 1986; Gianladis. U.S. Pat. No. 3,277,013, 1966; Rosano et al. U.S. Pat. No.3,778,381 (1973, Samejima et al. U.S. Pat. No.3,823,091, 1974, Yokoyoma et al. U.S. Pat. No.3,993,581, 1976; White, U.S. Pat. No. 4,366,169, 1982; and Arnaud and M. Mellul. FR No. 2688006A1, 1993. In the present application, we also disclose the use of the perfluorocarbon, Perfluorodecalin, to deliver oxygen to the skin using the Thixogel delivery system disclosed in this patent.

Still another application of the Thixosol technology is the delivery of a non-irritating antimicrobial agent derived from plants. One of these is Palmitoleic acid, a major fatty acid in Palm oil. Previously, it was found that human sebum contains a special isomer of Palmitoleic Acid (C16:1Δ6) that is a highly effective as an anti-bacterial agent against gram-positive bacteria. This isomer also acts synergistically with alcohols (Ethanol and Isopropyl Alcohol) and, thus, effective as a broad acting anti-microbial agent (Wille, 2003). This disclosure describes formulations incorporating Palmitoleic acid and other natural anti-microbials in the novel topical delivery system disclosed in this patent.

An important deficiency of many plant-derived ingredients with potential medicinal and cosmetic uses is they are only soluble in organic solvents or oils. These require a suitable vehicle for the delivery of oil-soluble plant actives. For this purpose, there are many botanical oils that might benefit from formulation in a hydrophobic delivery system such as that disclosed in this patent. These include: Almond, Avocado, Cottonseed, Olive oils, Corn, Menhaden, Safflower, Soybean, Peanut, Sesame, and polyunsaturated fatty acids. The latter contain linoleic acid, gamma-linolenic, acid, alpha-linolenic acid, and stearidonic acid; and omega-3- fatty acid, eicosoentanoic acid and docosahexaenoic acid. Other botanical extracts useful in personal care applications include: Lithospermum Offiinale (Napthoquinones), Thyme (Thymol), Hypercium Peforatum (Hypericin), Seabuckthorn (Carotenoids) Rosmarinus (Phyosterols), and Walnut(Juglone). Still other botanical extracts include: Ginger (Zingiberol, Gingerol) Chamomilla Recutita (α-Bisabolol), Carrot (β-Carotene) and Marigold- Calendula Officinalis (Calendulin, Quercetin). Special mention is made of oil derived from Sea Buckthorn berries, which have been successfully incorporated into our novel Thixogel formulations as disclosed in this patent.

In addition, a suitable delivery system is required for the delivery of plant-derived extracts enriched in polyphenols. Polyphenols possess well-known anti-irritants and antioxidant properties. Some polyphenol containing plant extracts that could benefit by formulation in the Thioxogel delivery system include: Grape seed, Honeysuckle, Cranberry and Green Tea (Camellia sinensis leaves). These plant materials are also rich in flavonoids (Quercetin and Apigenin), chlorogenic acid, and epigallocatechingallate and caffeine.

All of these agents have potent anti-oxidant activity could likewise be formulated in a hydrophobic-based skin care formulation. Similarly, plant extracts with putative anti-irritant properties derived from the following list are good candidates for incorporation into the Thixogel delivery system disclosed in this patent. They are: Aloe Vera, Chamomilla Recutia, Urtica Dioica, Betula Alba, Arnica Montana, Cinchona Persea Gratissima, Aloe Barbadensis, Chamomilla Recutia, Melissa Officinalis, Mentha piperita, Rosmarinus Officinalis, Fiscus Sabdariffa, Althea Officinalis, Artemisia bsinthium, Primula vulgaris, Salvia Officinalis, Santalum Album, Viscum Album, and Ilea Millefolium.

Depletion of antioxidants is known to cause oxidative damage to human skin (Podda et al, 1998). As discussed above, flavonoids are known to be potent anti-oxidants. Topical replacement of skin anti-oxidants may help to alleviate due to ultraviolet radiation and ozone exposure. Flavonoids require stabilization against oxidation by addition of co-reductants such as Vitamin E (α-tocopherol) or Vitamin C (Ascorbic Acid). No mechanism exists to reduce oxidized Vitamin E since there is no Ascorbic acid in the upper layers of the epidermis (stratum corneum). Lazendorfer et al., (2002) in U.S. Pat. No. 6,423,747 discloses cosmetic and dermatological preparations with favonoids having anti-oxidant properties. Illustrative examples mention standard\water-in-oil and water-in-oil formations without providing any evidence of their efficacy in these formulations.

Of particular importance to the category of polyphenols and flavonoids is the demonstration (Wille, 2003) that the mechanism of action for many plant-derived anti-irritants is their inhibition of protein tyrosine kinases associated with growth factor receptor stimulated autocrine control of cell proliferation that is the hallmark of many useful skin products that cause skin irritation, i.e., retinoic acid. The use of flavonoids as anti-irritants are among the plant-derived anti-irritants that are readily formulated in the novel hydrophobic delivery system claimed in this patent. They include many plants and herbs are rich in flavonoids as well as flavonoids present in Spanish Honeybee pollen. For example, rutin, quercetin, myricetin, and trans-cinnamic acid; all were present at >350 mg/100 g. Recently, it was reported (Bonina et al, 2002), that Kaempferol is the major flavonoid derived from lyophilized extracts of the flowering buds of capers (Capparis spinosa L). This material was shown to have both anti-oxidant and photo-protective effects in human skin.

In addition, there are many potential anti-irritant agents derived from fruits that can usefully be incorporated into the hydrophobic delivery system claimed in this invention. They include: Pyrus Malus, Camellia Sinensis, Ribes Nigrum, Vaccinium Macrocarpon, Citrus Grandis, Paulinia Cupana, Actinidia Chinesis, Citrus Aurantifolia, Citrus Aurantium, Carica Papaya, Passiflora Edulis, Prunus Persica, and Ananas Sativus.

### SUMMARY OF THE INVENTION

The compositions of the present invention may be used to formulate superior cosmetic and personal care products containing active ingredients, which have a shelf life of many years. The compositions of the present invention display thixotropic viscosity changes, i.e., they form semi-solids upon standing but which become pourable gels and lotions upon moderate mechanical agitation, an ideal characteristic of a lotion or gel in that it minimizes dripping but provides easy application. While providing ease of spreading on skin, the thixotropic microemulsions of the present invention provide long-lasting skin protection against water-borne chemicals and skin irritants.

The novel method for making the compositions of the present invention is simpler and less expensive than the method of making the Fantesk™ products. In addition, the starch-based microemulsions of the present invention do not form stiff layer on the skin, as the Fantesk™ product does, thereby providing a superior cosmetic vehicle, skin protectant, and vehicle for the delivery of hard to formulate plant actives. The compositions of the present invention also behave as a stable lotions for delivery of novel hydrophobic actives that provide anti-irritant and anti-aging activities. Hydrocolloid gel microemulsions of the present inventions are stable at least for three years at room temperature.

For the delivery of hydrophobic plant actives in cosmetic or personal care product formulations, the active must first be incorporated into the oil phase without traces of organic solvents. In addition, the final gel must be cosmetically acceptable. These issues have been solved by the composition and method of the present application by first dissolving the hydrophobic plant active in mineral oil. In addition, the delivery of hydrophobic active agents incorporated into composition is enhanced because not all of the oil phase is encapsulated by polysaccharide shells; the excess oil is thus available to carry hydrophobic solutes. The latter are able to penetrate into stratum corneum intercellular lipid upon application of the hydrocolloid gel emulsion into skin.

Cornstarch, a natural GRAS (Generally Recognized As Safe) plant polymer; which is inexpensive, may be used as a key ingredient of thixotropic microemulsions of the present invention. Cornstarch creates superior formulations because it is a good aqueous phase thickener with thixotropic and viscoelastic rheological properties, binds oil, forms a transparent “leave behind” protective film on skin, provides a greaseless, smooth feeling lotion/gel when formulated in oil-in-water emulsions, permits formulating microemulsion compositions over a wide range of starch: oil ratios, and forms a stable hydrocolloid gel emulsion with wide variety of natural and synthetic oils and combinations of different starches and oils. These characteristics make the cornstarch formulations of the pesent inventions widely useful for many cosmetic and drug delivery applications.

Of critical importance is the fact that Thixogel emulsions can be formulated with silicone oils. These oils reduce stickiness, improve spreading, lubricate, and improve the gloss of skin care formulation. They provide these attributes as well in the compositions of the present invention, and provide elegant skin feel when formulated in combination with starch, Petrolatum Jelly and Mineral Oil. While silicone oils are accepted by the CTFA as water proofing ingredients and for establishing skin protection claims, they are generally permeable to small molecules such as oxygen and water vapor. This deficiency is remedied in the thixotropic microemulsion compositions of the present invention by the addition of starch which upon drying forms films on skin that are impermeable to oxygen, and by the addition of mineral oil and petrolatum, which are impermeable to water vapors.

A common issue with any starch-based lotion is its drying effect on skin. In the compositions of the present invention, this can be overcome by the addition of a moisturizing agent or humectant. Glycerol, a well-known and widely accepted cosmetic humectant may be easily incorporated therein. As a poly (alcohol), it has a strong affinity for water and is well tolerated on skin even at high concentrations. Other sugars such as Trehalose, and sugar esters, can also be used as cosmetic humectants in the thixotropic microemulsion compositions of the present invention. Natural moisturizing factor (NMF, pyroglutamic acid) may also be employed either alone or in combination with glycerol.

While starch is subject to both bacterial and fungal degradation, unpreserved starch in the thixotropic microemulsions of the present invention can be protected using natural preservatives such as Tea Tree Oil and CITRICIDAL, as they are readily incorporated into the oil phase ingredients. CITRICDAL, an oil from grapefruit seeds is also a very effective antimycotic agent. The use of low levels of Benzalkonium Chloride as an emulsifier also serves the dual purpose of inhibiting the growth of both bacteria and yeast. Thixogel starch formulations containing both Benzalkonium Chloride (0.5%) and CITRICIDAL (0.5%) have remained uncontaminated for several years.

The hydrophobic plant actives derived from carrot extracts (carotenes) and tomato. paste (lycopenes) are especially important in the compositions of the present invention. Both actives are known anti-cancer agents and regulators of epithelial homeostasis. For both materials, extracts have been prepared and enriched by sequential extraction with ethyl alcohol and organic solvents. These extracts are then dried to a powder form and solubilized in Mineral Oil, preferably containing Vitamin E (0.1%) to prevent the photo-bleaching that occurs in mineral oil solutions exposed to visible light and oxygen. FIG. 1 presents results of studies showing the photo-protective effect of Vitamin E on photo-bleaching properties of lycopenes. Similar results have been obtained with the addition of Vitamin E to prevent the photo-bleaching of carotenes derived from Carrot Extracts.

Perhaps, the most interesting plant active formulated in thixotropic microemulsion delivery systems of the present invention is an extract of corn tassels identified by HPLC methods as largely comprised of esters of phenoxyacetic acid, a potent anti-irritant.

It is an object of the present invention to produce cosmetically acceptable lotions and gels using natural starch and oils to create a vehicle for the delivery of “natural products” with potential to incorporate hydrophobic plant -derived actives in the cosmetic and personal care markets. It is a further object of the invention to produce a composition for use in cosmetic and personal care product formulations which makes it easy to incorporate plant-derived hydrophobic anti-irritants. These objects, as well as other objects which will become apparent from the discussion that follows, are achieved, in accordance with the present invention, which comprises an oil-in-water, thixotropic microemulsion composition, and a method for making the same, and drug delivery systems, or “active ingredient” delivery systems, made from said composition, as set forth immediately below.

The present invention is a composition comprising a thixotropic microemulsion. The essential ingredients of the emulsion are starch (from about 1% yo about 4%), water, an emulsifier, and from about 1 to about 20% oil. The starch usable in the invention are natural, hydrophobic starches or chemically modified starches, and polysaccharides, and combinations thereof. The composition has been shown to be a stable oil-in-water emulsion, with more than three years stability at ambient conditions. The average particle size distribution of the microemulsion is clustered around about 0.5 to 3 microns. The average particle size distribution of the microemulsion is clustered around about 0.3 to 5 microns, with average particle size of the microemulsion is 0.8 microns. The molecular weight of the thixotropic microemulsion composition of the present invention is greater than about 300,000 daltons.

The starch is selected from the group consisting of corn starch, potato starch, and certain polysaccharaides. The surfactant, selected from the group consisting of anionic surfactant, such as sodium lauryl sulfate, or a cationic surfactant, such as benzalkonium chloride, or a non-ionic surfactant, such as Triton X-100. When the emulsifier is sodium lauryl sulfate, the starch:oil ratio can be from about 1:3 to 3:1 The composition may further include a humectant, such as glycerol, Trehalose, and sugar esters, or combinations thereof. The oil utilized in the composition may be silicone type oils, preferably dimethicon and decamethylcyclopentasiloxane. Other oils which may be used in forming the thixotropic microemulsion of the present invention are mineral oil, berry wax, petrolatum jelly, paraffin oil, polydimethylsilicone oil, perflurodecalin, and vegetable oils, and combinations thereof.

The composition of the present invention is an ideal delivery vehicle for an active agent. Natural hydrophobic plant extracts which are anti-irritants to the skin, may be easily incorporated into the composition of the present invention, to create a vehicle for the delivery of many desired “active agents” which are skin irritants. One such “active agent” which may be incorporated into the composition of the present invention is the oxygen carrier is perflurodecalin. A preservative, especially natural preservatives such as Citricidal and Tea Tree Oil, may be easily included in the composition.

The thixotropic microemulsions of the present invention may be used to make topical gel drug delivery systems. For instance, an anti-irritant may be easily added to the thrixotropic micoremulsions, to minimize the skin reaction to drugs, or active ingredients in the delivery systems. An example of the natural anti-irritants that may be easily incorporated into the thixotropic microemulsions of the present invention are hydrophobic plant extracts and hydroalcoholic plant extracts. Another anti-irritant which may be easily incorporated into the microemulsion is Bisabol.

Some preferred plant extracts are those from Autumn Olive berries, corn tassel, grapefruit seed oil, Sea Buckthorn oil, green onion leaves, red swiss chard, red seedless grapes green tea leaves, hops (Humulus Lupulus), dried catkins from Linden trees (Tilia sp.), dried catkins of Oak Trees (Quercus, sp.), dried flowers of Lavender (Lavendar), dried cocoa powder, and dried cinnamon powder. Fruit extracts are especially preferred.

Examples of naturally irritating “active ingredients” which may safely be included in a delivery system of the thixotropic microemulsion with anti-irritant of the present invention are AHA's, retinol, and Palmitoleic Acid.

The stable oil-in-water, thixotropic microemulsions of the present invention are made by a novel process emulsion, comprising:
A) mixing a starch in cold water to form a slurry, and adding a minor amount of an emulsifier,
B) heating and stirring the mixture until the starch undergoes thermal melt, and the mixture is clarified,
C) ceasing to heat the clarified mixture, and cooling it to a temperature just above the melting point of the oil to be emulsified, and
D) using low shear mixing, adding oil to the clarified mixture to form a thixotropic microemulsion, comprising from about 1-4% starch and from about 1 to about 20% oil.

The starches used successfully in the method are natural, hydrophobic starches, chemically modified starches, polysaccharides, and combinations thereof. Fragrance may be easily added to the thixotropic microemulsion, preferably in step D Vegetable colorings may be easily added to the cold water in step A. Citricidal may be easily added to the mineral oil in step A. Palmitoleic acid, in alcohol, may be easily added to the mixture in step A.

The stable oil-in-water thixotropic microemulsions of the present invention may be used to create a protective layer on the skin of the hands; a flexible glove-type coating, or a “glove within glove”. As the microemulsions of the present invention are alcohol resistant and moisturizing, a flexible skin layer formed by applying the microemulsions will tolerant multiple rinsings with alcohol, while maintaining the moisture of the underlying skin. Thus the skin layer formed by the microemulsion is a sanitary layer, which may be rinsed in alcohol. As may be easily understood, the microemulsions, when applied to the skin create a wound dressing skin layer, which may contain an anti-bacterial. Such wound dressing would find easy application as stoma dressings.

For a full understanding of the present invention, reference should now be made to the following detailed description of the invention and its preferred embodiments, and the accompanying figures.

### BRIEF DESCRIPTION OF THE DRAWINGS

FIG. 1A is a graph illustrating the effects of photobleaching of lycopenes dissolved in mineral oil, over time.

FIG. 1B is a graph illustrating the photo-protective effect of Vitamin E (0.1% in Mineral Oil) on the photobleaching of Lycopene prepared from tomato paste.

FIG. 2 is is a graph illustrating the effect of three Thixogel type formulations: formulation 1(DermSeal), formulation 2 (EktaSeal), and formulation 5 (UltraDerm) on elevation of skin hydration over time.

FIG. 3 is a photographic perspective view of volar arm stains illustrating the protective effect of applying Vaseline (A), Thixogel formulation 1 (B), or (C) no lotion, on volar arm skin, stained after these applications with a solution of crystal violet stain and washed with water to remove excess stain

FIG. 4 is a photographic view illustrating the protective effect afforded by application to aluminum foil of Thixogel Formulation 1 against the corrosive effect of 3N Hydrochloric Acid

FIG. 5 is a photographic top view of the effect of air-drying and rehydration with water on the reversible uptake of water and gel consistency of Thixogel formulation 5.

FIG. 6 is a graph showing the kinetics of oxygen release over time, for various oxygen charged formulations: water (,), water plus 10% PFC (▪), starch (4%, ◯), starch (4%)+PFC (▴), and OxyTega (●).

### DETAILED DESCRIPTION OF THE INVENTION

The term Thixogel, as used herein, describes an oil in an aqueous starch thixotropic microemulsion, which has been differently formulated, to provide ease of formulating plant-derived hydrophobic anti-irritants. Most creams and lotions in the cosmetic market are emulsions. They may exist as microemulsions, multiple emulsions, fluorocarbon emulsions, and gel emulsions. All Thixogel formulations are oil-in-water emulsions. More than a hundred Thixogel-based formulations have been made and tested to date. The basic ingredients of all Thixogel formulations are water, starch, oil, and an emulsifying agent. To this basic formula one may optionally include a humectant, one or more silicone type oils, an active agent such as an antimicrobial, hydrophobic plant actives, and natural preservatives. Among these four basic components many different natural and modified starches, many natural vegetable and synthetic oils, and anionic, cationic and non-ionic surfactants have been formulated. A common feature of all formulations is the stability of starch-oil dispersions formed by heating and mixing starch and oil under controlled temperature and mixing conditions. Thixogel formulations are so-called because they display thixotropic viscosity changes, i.e., they form semi-solids upon standing but with become pourable gels and lotions upon moderate mechanical agitation.

Thixogel is a novel oil-in-water type hydrocolloid emulsion comprised of starch, water, oil, and an emulsifying surfactant. The starch may be a natural starch, or a modified starch. The oil may be paraffin oil, mineral oil, polydimethylsilicone oil, perfluro-decalin, vegetable oils, and various combinations of these oils. The emulsifying agent may be an anionic, a cationic or a non-ionic type of surfactant.

Thixogel is formed by a simple two step process. Simply, a cornstarch slurry is prepared in cold water containing a specified low concentration of the surfactant and, if desired for moisturization, a specified amount of glycerol. The mixture is heated with continuous stirring until all of the starch is dissolved. The clarified starch is removed from heat, and allowed to cool to 65° C., when the oil phase ingredients are blended in the aqueous phase by low shear mechanical mixing. It is, therefore, apparent that control of both temperature and mixing conditions are essential for reproducible and consistent formulation.

This starch-based emulsion has proved to be superior as a cosmetic vehicle, a skin protectant, and a vehicle for the delivery of hard to formulate plant actives. It also behaves as a stable lotion for delivery of novel hydrophobic actives that provides anti-irritant and anti-aging activities.

Hydrocolloid gel emulsions are stable at least for three years at room temperature. The viscosity of such systems are a function of the starch to oil ratio. Low viscosity liquids are usually formed when the starch concentration is below 1% in such systems. Gels are seen at concentrations of starch in the range of 1% to 4%, and at oil concentrations below 10%. Starch-oil dispersions are achieved by processing at temperatures above 75° C. and are stabilized by high speed mechanical blending in the presence of low levels of a surfactant. Scanning electron microscope pictures of Thixogel systems reveal the presence of oil droplet within a starch matrix with an average size distribution clustered around 0.5-3 microns. Previous studies have shown that the oil droplets in Thixogel system are coated with a polysaccharide shell. This shell prevents coalescence of the oil droplets and ensures emulsion stability due to two forces. The tendency of high molecular weight polysaccharide such as starches to precipitate due to their low water solubility, and the favorable systems increase in entropy and energy reduction that occurs when the starch molecules precipitate and form a carbohydrate layer on the oil droplets at the water-oil interface.

Thixogel gels are soluble in the hydrocarbon oil phase layer that develops on the surface of the skin This hydrocarbon oil phase results from mechanical breakdown of the polysaccharide shell surrounding the oil droplets As a result of this mechanical breakdown there is a release of oil droplets, which then coalesce and rapidly form a continuous oil Thixogel is considered to be a poor delivery vehicle for hydrophilic cosmetic actives. Although, Thixogel formulations may be they are readily incorporated into the bulk water phase and agent soluble only in the water phase may be released on the skin surface by zero-order kinetics. They are not able to penetrate the stratum corneum by means of passive diffusion.

As noted above, changes in starch concentration directly affect the viscosity of the formulation. Low starch concentrations fail to produce a stable emulsion, while concentrations above 4% result in thick unusable gels. Within the range of useable starch concentrations, the concentration of oil phase ingredients also affect rheological properties. Oil concentrations below 1% produce watery emulsions with an oily skin feel. High oil concentrations above 12% are less stable and require increased levels of emulsification with undesirable skin associated reactions.

Batch performance was certified by viscosity measurements made with a Brookfield Thermosel instrument. Thixogel formulations made with DRY-FLO/AF® starch (4%) and Mineral Oil (8%), and Benzalkonium Chloride(1%) had a viscosity of 13, 200±100 cps at 20° C. and 2.5 rpm with a #27 SPDL blade. Under the same test condition a Thixogel formulation with 3.3% DRY-FLO/AC® starch, 10% Petrolatum, and 0.5% Benzalkonium Chloride had a viscosity of 8,500±100 cps.

Stable emulsions can be also formed by heating starch solids in the presence of Sodium Lauryl Sulfate (0.5%) or Triton X-100 (0.1%) at starch: oil ratios of 1:1, 1:2, 1:3, and 3:1. The gelatinized starch solubilized in an aqueous surfactant solution is then blended with paraffin oil using either natural starches, e.g., Pure Food Grade Powders, and Wax starch, or modified starches, e.g., StaMist-365, Solance and DRY-FLO. Stable gel emulsions can also be formed as above by heating the starch above 75° C. and blending in mineral oil at starch: oil ratios of 1:1 and 1:2. This can be accomplished using Pure Food Grade Powders or Waxy maize type starch. Finally, stable gel emulsions can be formed by heating the starch above 75° C. in the presence of Benzalkonium Chloride (1%) at a starch: oil ratio of 1:1 (using either Pure Food Grade Powders or a hydrophobic starch e.g., StaMist-365). In special cases, lecithin can be substituted for Benzalkonium Chloride at a starch: petrolatum ratio of 1:2.

Polydimethylsiloxane fluids (viscosity range 10,000 to 60,000 cps) can be substituted for petrolatum at starch:581 oil ratios of 1:1 and 1:2 in the presence of Benzalkonium Chloride (1%), and in another formulation where an oxygen-carrying oil was required, perfluorodecalin, was substituted for petrolatum at a 1:2 starch: oil ratio in the presence of 1.6% Benzalkonium Chloride. All of the above formulations are generally useful as skin protectant gels. However, they may be made into moisturizing gels by simply incorporating 10 to 20% glycerol in the aqueous phase ingredients phase prior to heating and blending with the oils.

The basic Thixogel starch: oil-in-water hydrocolloid emulsions provide ease of spreading on skin, and long-lasting skin protection against water borne chemicals and skin irritants. More sophisticated formulations are required for the delivery of hydrophobic plant actives. In such cases, the active must first be incorporated into the oil phase without traces of organic solvents. In addition, the final gel must be cosmetically acceptable. These issues have been solved by first dissolving the hydrophobic plant active in mineral oil. The delivery of hydrophobic active agents incorporated into Thixogel is enhanced because not all of the oil phase is encapsulated by polysaccharide shells; the excess oil is thus available to carry hydrophobic solutes. The latter are able to penetrate into stratum corneum intercellular lipid upon application of the hydrocolloid gel emulsion into skin.

For example, carrot extracts enriched in α& β-carotenes_are totally insoluble in water. Likewise, tomato paste-derived lycopenes_are also completely insoluble in an aqueous phase. To obtain such materials as active ingredients, they first must be extracted into an organic solvent such as hexane, and thereafter dried to a powder from which they may be solubilized in mineral oil. This technique is highly useful because it allows formulation of such materials directly into the oil phase ingredients of Thixogel. In addition, Dimethicone (DC-200 Fluid) and Decamethyl cyclopentasiloxane (DC-245 Fluid) are added to the oil phase ingredients to obtain a smoother skin feel. In the formulation discussed in the examples, Thixogel gels have been employed as a hydrophobic delivery system to deliver other plant-derived actives including retinoids, and flavonoids.

Natural starches are the key ingredients of this invention. They are high molecular weight (>1-2×10⁶ Daltons) polysaccharide polymers. Pure food grade starch is composed of 70% amylose and 30% amylopectin. A natural corn mutant variety produces an amylose-free starch known as Waxy cornstarch. In general, natural starches are not soluble in cold water and must be heated above 70° C. to make them dissolve completely. Upon cooling, natural starches at concentrations above 2% solids, form semi-translucent rigid gels. These are referred to as pre-gelatinized starch. Gel formation in natural cornstarch is due to the rapid re-association of upon cooling of intra- and inter-molecular hydrogen bonds between different polymer side chains. Once starches are subjected to a heating and cooling cycle in water, and gel formation begins, they are too rigid and sticky to be used directly on skin. For this reason such starches are to be considered poor candidates as bulk carrier for personal care actives.

Natural starches are known to have a limited capacity to bind oils and as such, they have been widely used as thickeners and emulsifiers in the food industry. Finely milled adheres well to skin and like talc possess an slippery skin feel. Indeed, starches have a long and venerable place in the personal care market as a basic ingredient of dusting powders for babies. Modified starches have also been used as aqueous rheology modifiers and as bulk carriers in protective skin care lotions. Natural cornstarch including Waxy cornstarch are preferred ingredients relative to modified starches. They form high yield stress gels and are better thickeners than modified starches because they have a higher molecular weight. Such natural starches with larger molecular sizes will encapsulate dispersed oil droplets more readily than low molecular weight degraded starch molecules. This phenomenon has been demonstrated by hydrolyzing natural cornstarch by boiling in 1N Sodium Hydroxide, recovering the treated starch, and demonstrating that it had lost its ability to form a hydrocolloid gel emulsion (with oil) in the presence of an emulsifier.

We have chosen natural cornstarch as a key ingredient of Thixogel formulations because it is: a natural GRAS (Generally Recognized As Safe) plant polymer; an inexpensive ingredient (bulk price <10 cents/pound), a good aqueous phase thickener with thixotropic and viscoelastic rheological properties, bind oil, forms a transparent “leave behind” protective film on skin, provides a greaseless, smooth feeling lotion/gel when formulated in oil-in-water emulsions, formulations can be made over a wide range of starch: oil ratios, forms a stable hydrocolloid gel emulsion with wide variety of natural and synthetic oils and combinations of different starches and oils. Therefore, such systems are widely useful for many cosmetic and drug delivery applications.

The choice of oil to form hydrocolloid gel emulsions with starch presents no serious limitation. Saturated hydrocarbons such as Mineral Oil and Petrolatum Jelly, which are standard ingredients for formulating emulsions in the cosmetic industry, readily form stable Thixogel emulsions in the presence of low levels of emulsifying agents. Indeed, natural fatty acids have been substituted for common oils as the “oil phase”. Examples include: oleic acid, a major constituent of soybean oil, and linoleic acid, present in olive oils. Another natural oils that form Thixosol emulsions having unique benefits for skin is Meadowfoam Oil. This oil has a virtually pure C22- chain length hydrocarbon compound. Finally, starch oil-in water emulsions have been prepared using Triacetin®, a natural triacetate glyceride. Triacetin forms a stable emulsion in the presence of Benzalkonium Chloride (1%) or Sodium Lauryl Sulfate (0.5%), but not with Triton X-100 (0.1%).

Of critical importance is the fact that Thixogel emulsions can be formulated with silicone oils including a high viscosity Dimethicone (60,000 cp), and less viscous Poly(dimethyl)siloxane (200® Fluid), and Dimethyl cyclopentasiloxanes (245® Fluid). These oils reduce stickiness, improve spreading, lubricate, and improve the gloss of skin care formulation. They accomplish these attributes as well in Thixogel formulations and provide it with elegant skin feel when formulated in combination with starch, Petrolatum Jelly and Mineral Oil.

The development of topical delivery system for plant-derived actives was greatly aided by modifications of the Thixogel starch: oil hydrogel emulsion. Studies have shown that hydrophobic plant-derived actives can be formulated into the mineral oil phase of Thixogel emulsions. Among the hydrophobic plant actives so formulated are anti-oxidants such as flavonoids, beta-carotene, and lycopenes. Other active agents include retinoids, e.g., retinyl pamitate, Vitamins A, E, and K, plant sterols, and a variety of medicinal and herbal oils, e.g., Sea Buckthorn oil.

As noted previously, natural starches are not composed entirely of pure polysaccharide -polymers. Generally, there are small amounts of protein and phospholipids. Thus, and while they are inter-facially active, they are not present in sufficient quantities to provide commercially stable emulsions, which may act as endogenous emulsifying agents. However, starch, by itself, is a poor emulsifying agent, and an emulsifier must be added to form stable starch oil-in-water dispersions.

We have chosen to prepare starch-in- oil dispersions by formulating the starch ingredient in an aqueous surfactant solution. As mentioned earlier, useful surfactants may be anionic, cationic or non-ionic amphoteric compounds. These are present typically at concentrations below 1%(Wt. %). Most Thixogel formulations use the cationic emulsifier, Benzalkonium Chloride. In principle, the anionic surfactant, Sodium Lauryl Sulfate, and the non-ionic surfactant Triton X-100, were useful in forming Thixogel emulsions, but the former is a well known skin irritant, and the latter is not a very effective anti-microbial agent. A possible difficulty in using Benzalkonium Chloride in Thixogel formulations is its ability to form ion-pairs with fatty acid compounds. This does, in fact, occur if Benzalkonium Chloride and fatty acids are heated together during processing. However, the ion-pair formation may be prevented either by adding the fatty acid during the cooling phase and after emulsification has occurred, or by holding the pH at or above pH 6.

The natural phospholipid, Lecithin, is also a good emulsifier for Thixogel systems. It can be formulated as a 5% ethanol solution with pure food grade cornstarch and forms a stable hydrocolloid gel emulsion with Petrolatum at a starch:oil ratio of 1:2.

Starch is subject to both bacterial and fungal degradation. Unpreserved starch in Thixogel emulsions are mostly subject to contamination by molds. Several effective, all-purpose, natural preservatives are Tea Tree Oil and CITRICIDAL, Tea Tree Oil has recently been shown to be an effective antimicrobial agent for veterinary applications. It is readily incorporated into the oil phase ingredients of Thixosol formulations. Likewise, CITRICDAL, an oil from grapefruit seeds is a very effective antimycotic agent. CITRICIDAL appears to be superior to Tea Tree oil t because it is less volatile and aromatic than Tea Tree Oil. Thus, it is more long lasting as a preservative.

The use of low levels of Benzalkonium Chloride as an emulsifier also serves the dual purpose of inhibiting the growth of both bacteria and yeast. Thixogel starch formulations containing both Benzalkonium Chloride (0.5%) and CITRICIDAL (0.5%) have remained uncontaminated for several years.

Natural ingredients, botanicals, medicinal herbs and plant oils have all drawn significant attention to formulators in the cosmetic industry. In particular, Vitamin E is present in many skin care products as a fat-soluble antioxidant, and as a possible anti-aging agent. Ascorbic acid (Vitamin C), which is derived from citrus fruits, is often included in skin care formulations. However, this water-soluble vitamin is very unstable in aqueous solutions. To overcome this deficiency, Vitamin C is often paired with another redox partner such as the Tocopherols (Vitamin E) in order to slow its oxidation. Another recent and useful approach is the use ascorbic acid is in the form of a fatty acid ester such as Ascorbyl Palmitate. Ascorbyl Palmitate has been chosen for use in Thixogel formulations and is typically incorporated into the Mineral Oil phase. Preliminary in silico experiments showed that it could be slowly released from starch-oil emulsion matrix and act as anti-oxidant.

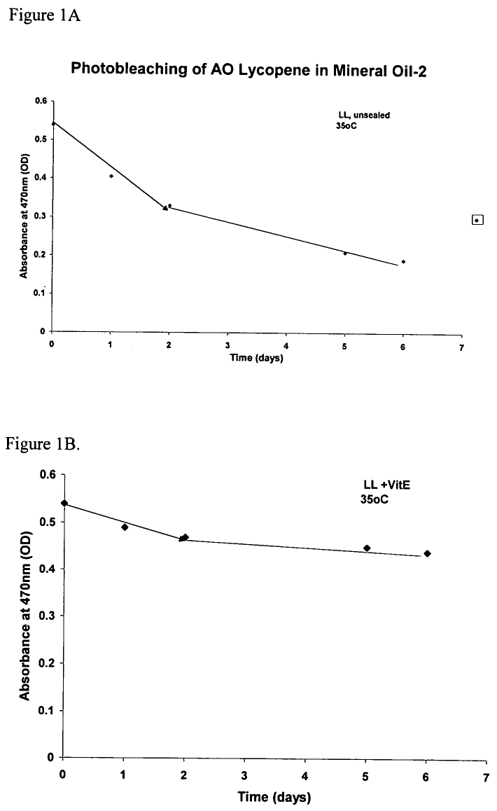

The principal plant actives under development are the hydrophobic plant actives derived from carrot extracts (carotenes) and tomato paste (lycopenes). Both actives are known anti-cancer agents and regulators of epithelial homeostasis. Their role in epidermopoeisis and skin care is still under investigation. For both materials, extracts have been prepared and enriched by sequential extraction with ethyl alcohol and organic solvents. These extracts are then dried to a powder form and solubilized in Mineral Oil. A clear necessity for the preparation of such formulations is to prevent the photo-bleaching that occurs in mineral oil solutions exposed to visible light and oxygen This may typically be accomplished by addition of mineral oil solutions of Vitamin E (0.1%). FIG. 1 presents results of studies showing the photo-protective effect of Vitamin E on photo-bleaching properties of lycopenes. Similar results have been obtained with the addition of Vitamin E to prevent the photo-bleaching of carotenes derived from Carrot Extracts.

Perhaps, the most interesting plant active formulated in Thixogel delivery systems is an extract of corn tassels. The story behind this ingredient began -several years ago, when the author was driving to work along a country road on a warm August day. The car window was down, and at a particular spot along the road, there came a strong aroma, slightly reminiscent of the pungent odor from blossoms of the Mimosa tree. Later, the Inventor was able to determine that the smell came from cornfield, and, in particular, from the corn tassels. Sometime later, the corn tassels were removed and extracted with methanol. The aromatic material was identified by HPLC methods and seen to be largely comprised of esters of phenoxyacetic acid. This observation was intriguing, because previous studies by the inventor had shown that phenoxyacetic acid was a potent anti-irritant.

Subsequently, new studies have confirmed the earlier work. The corn tassel active material is now called Tasselin. Hydroalcoholic extracts of Tasselin can be concentrated to an oil by rotary evaporation followed by dissolution into Mineral Oil. This solution can then be added later to the oil phase ingredients of Thixosol delivery system.

The choice of an oil or combination of oils is solely determined by the application. Petrolatum is a good skin protectant and provides some moisturization relative to Mineral Oil and other vegetable oils. Likewise, the use of very viscous silicone oils can enhance skin protectant. Thixogel formulations when combined with Petrolatum. Less viscous lotions and gels could also be are formed by using liquid oils such as Mineral Oil and vegetable oils as the oil-phase ingredients. Soybean Oil, for example, provides Oleic Acid, which is known to enhance skin permeation of hydrophilic drugs. It is believed that the oleic acid helps to fluidize the lipid bilayer of the intercellular epidermal lipids in the stratum corneum.

Another useful oil is linoleic acid present in linseed and cottonseed oils. Yet, another useful oil is olive oil, which contains omega-3 fatty acids. These fatty acids are known to be antibacterial and can help repair the damaged lipid skin barrier perturbed by dietary insufficiency in essential fatty acids (i.e., linoleic acid) present in essential fatty acid deficiency disease.

Artificial sebum, a mixture of ceramides, fatty acids, triglycerides, cholesterol, cholesterol esters and squalene, could also be substituted for the oil phase ingredients in Thixogel formulations. It was previously reported (Wille and Kydonieus, 2003) that palmitoleic acid is the most active anti-microbial fatty acid present in sebum. This fatty acid has been used in Thixogel formulations in order to design a grease-less, spreadable antibacterial hand gel.

The principle action of a surfactant is to reduce the size of the oil droplets. The other being stabilization of those droplets relative to their tendency to coalesce. This process increases interfacial surface area available for interacting with amphoteric emulsifying agents. Anionic surfactants, with a net negative charge, are well known to interact with phospholipids in the living membrane of cells and, thereby, destabilize the lipid bilayer. Such surfactants are among the most skin irritating detergents present in many Shampoo formulations.

As described previously, the cationic surfactant, Benzalkonium Chloride was chosen for most Thixogel applications and formulations described in this chapter. It is approved for use as a preservative for certain external uses. Starch-in-oil type Thixogel formulations containing 0.5% Benzalkonium Chloride have been found to be only marginally irritating in a standard ocular irritancy test, and no skin irritation was observed when applied as a single dose application to human skin in a panel of human volunteers

In order to determine what surface and interfacial science principles are involved in the Thixogel delivery system, many studies have been carried out to uncover the role of various emulsifiers in producing stable hydrocolloid gel emulsion. While it is known that starch powders can, themselves, absorb oil. The amount of oil absorbed by natural starch powders is very limited, indeed.

Hydrophobically-modified starches such DRY-FLO/AC® can readily be dispersed in an oil phase. However, these starch granules remain in the oil phase when mixed with water unless an emulsifying agent is present. It is, of course, known that oil and water do not mix unless the interfacial tension between them is minimized. The required work of dispersion for an oil phase in water, placed into in an oil-in-water emulsion is lowered by reducing interfacial tension.

Macromolecular polymers such as proteins, e.g., gelatin solution, are known to reduce the surface tension of water. In like manner, molecules like phospholipids will reduce the overall interfacial tension between oil and water. It is proposed that phospholipids occur at the interface between oil and water and thereby cause an increase in ordering of water molecules at the oil-water interface. The phospholipids provide polar-polar interactions between the polar groups of water and the polar head group of the phospholipid. By reducing the degrees of freedom of the polar substituents the overall energy of the system is reduced, and thereby interfacial tension is reduced. By the same token, emulsification of oils is aided by the apolar side chains of the phospholipid. These interact with the apolar groups at the surface of the oil phase, thereby reducing the degree of freedom of the oil molecules. The result is a lowering the entropic contribution of the phospholipids, and creation of a more ordered system, as seen in self-assembling phenomena seen in the lipid bilayers and micelles. It should be noted here that natural starches contain up to a few percent of natural phospholipids, This fact may help to explain their oil-binding properties of natural starches.

In the case of Thixogel, preparation of the hydrocolloid gel emulsion is accomplished by adding an surfactants and sufficient mechanical mixing such that there is an increases in surface area through oil droplet size reduction. Stabilization of these emulsions is accomplished by interaction of starch molecules with the oil droplets, perhaps, by means of the phopholipids contained in semi-purified natural starches, that act as “emulsifiers.” Deposition of starch molecules at the oil interface occurs because the large starch molecules (>10⁶ Daltons) are less soluble in the water phase and by interacting with oil molecules reduce the overall interfacial energy of the system

Thixogel Personal Care delivery systems can affect the delivery of active components and control the release rate from the vehicle by interacting with the active agent. Such systems may also incorporate permeation enhancers. These are capable of altering the flux of the active through the stratum corneum or may, in fact, enhance hydration of the stratum corneum. For example, as shown in the examples, oleic acid has been combined with palmitoleic acid alone, or in combination with ethanol as vehicle components that can alter the rate of movement of active agents through the stratum corneum.

### DESCRIPTION OF THE PREFERRED EMBODIMENTS

The preferred embodiments of the present invention will now be described.

### Example 1

### Emulsification Studies on Thixogel Formulations

Table 1 presents a summary of results evaluating the ability of various surfactants, fatty acid and oils to form stable Thixogel type emulsions. The evaluation was undertaken to determine ways to reduce the percentage of Benzalkonium Chloride, which can lead to skin irritation. Each row represents a formulation (A-G) which was positive for emulsification by demonstrating incomplete separation of phases at 60 minutes.

TABLE 1

| | BC | OleicAcid (Wt %) | C | 20% DRY-FLO |
|---|---|---|---|---|
| A | 1.0 | 0 | 0 | None |
| B | 1.0 | 0 | 0 | Added |
| C | 0.5 | 1.0 | 0.5 | None |
| D | 0.5 | 5.0 | 5.0 | None |
| E | 0.1 | 0.5 | 0 | Added |
| F | 0.1 | 0 | 0 | Added |
| G | 0 | 1.0 | 0.5 | None |

Combinations of DRY-FLO Starch, Oils and Surfactant that Emulsify a Two-Phase Oil and Water Mixture.
BC = Benzalkonium Chloride(Wt %), C = Citricidal (Wt %)
Note:
The assay system consisted of 2.0 ml of soybean oil layered on top of 2.0 ml of water in a test tube. Each of the combinations above in each row was added at zero time and test tubes shaken to thoroughly mix the phases. The test was considered positive for emulsification if there was incomplete separation of phases at 60 minutes.

As shown by the result in row A, 1.0% Benzalkonium Chloride(BC) emulsifies the soybean oil and water of the assay system. Row B indicates that 1.0% BC was also effective to create a stable emulsion when 20% DRY-FLO modified starch was added, but 0.1% of BC, together with the DRY-FLO was sufficient to create an stable emulsion. Benzalkonium Chloride(BC) at concentrations below 1% was ineffective to emulsify the assay system, unless combined with oleic acid at 0.5% or higher, as shown in rows C and D. Though DRY-FLO is known to absorb oil, DRY-FLO alone, without BC was insufficient of emulsify the assay system. However, the combination of BC and DRY-FLO (20%) is capable of forming a stable emulsion of soybean oil and water, even at 0.1% Benzalkonium Chloride, as shown in row F. Lastly, the combination of Oleic Acid (1%) and CITRICIDAL (0.5%) was found to be effective in producing stable oil-in-water emulsion. By itself, Oleic Acid was found to be ineffective at stabilizing the emulsions. To a limited extent, addition of 0.5% CITRICIDAL can also lower the concentration of required emulsifiers.

When adding active ingredients to the micoremulsion of the present application, special processing may be required, as ion-pairs may be formed between anion and cationic ingredients when heated during the pre-gelatinization step. This can often be avoided by altering the pH. One combination of cationic and anionic ingredients successfully combined was 0.5% Palmitoleic Acid and 0.1% Benzalkonium Chloride.

In another series of investigations, the ability of several formulations to act as emulsifiers, themselves, were examined. In this assay, 0.2 ml of each formulation was added directly to a test tube containing 2.0 ml of Soy Bean Oil carefully layered on top of 2.0 ml of water. The mixture was then shaken to thoroughly mix the two phases. A control containing just the two phases was used as a reference for measuring the extent of emulsification and the resulting stability of the emulsions. Addition of 0.2 ml of a Thixogel formulation containing Starch (DRY-FLO/AC®) and Mineral Oil in a 1:2 ratio, in the presence of 0.5% or more of Benzalkonium Chloride, led to the appearance of an interface after 60 minutes. A transfer of 20% of the water phase into the oil phase was accomplished with this approach. In a second formulation, Starch (DRY-FLO/AC®) and Petrolatum (in a 1:2 starch: oil ratio) in the presence of 0.1% Benzalkonium Chloride, and 0.5% Palmitoleic Acid generated only a 10% shift if water into the oil phase. Finally, a similar formulation, containing 4% Dimethicone, produced a 5% transport of oil into the water phase. These results suggest that emulsification can be brought about by both movement of oil into the water phase and by movement of water into the oil phase of such systems.

### Example 2

### Skin Hydrating Thixosol Formulations

The following five formulations (DermSeal-#1, Aqua Seal-#2, VegaSeal-#3, EktaSeal-#4 and EktaDerm-#5) are basic skin barrier gels and lotions that possess good skin protectant and skin moisturizing properties. These model formulations have been tested by a variety of tests including skin hydration using a device that measures skin capacitance, the Corneometer (Model CM 825, Courage & Khazaka, Koln, Germany).

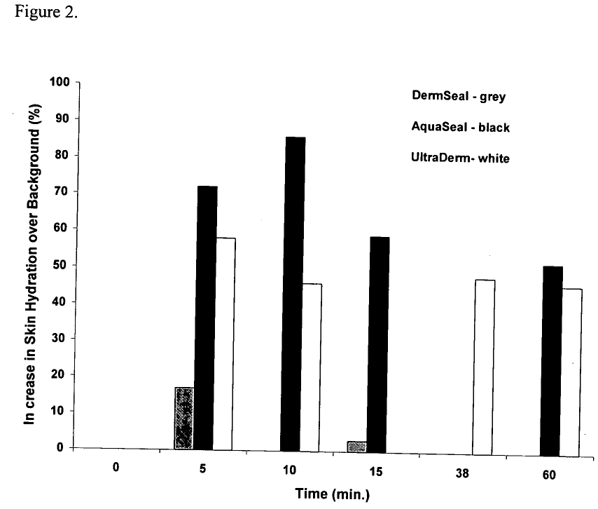

FIG. 2 shows that Formulation 1 has virtually no effect on skin hydration, while Formulation 2 significantly elevates skin moisture to levels 50% greater than that seen in normally hydrated skin. The elevated skin moisture obtained persisted for at least one hour after application of this formulation at 26° C. and a relative humidity of 28%. Similarly, Formulation 5 with 10% Glycerol provides significant elevation of skin moisturization.

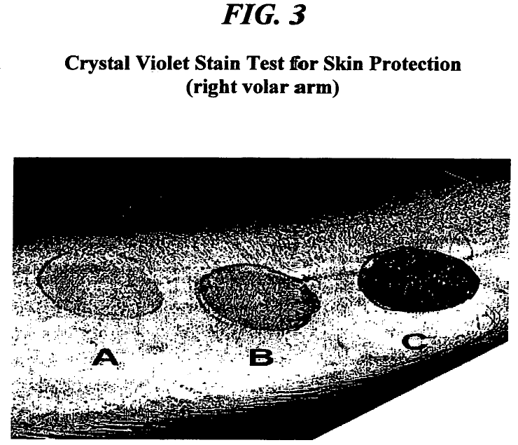

The skin-protecting effect of Formulation 1 was demonstrated by the crystal violet stain test as describe here. Several 2.5 cm² circles are drawn on the volar arm surface of a human subject. The encircled areas are then coated with test material (A, Vaseline; B, Formulation 1 (Tx-1D), or C) no coating material (unprotected control). Discs of filter paper are then dipped into a 0.2% crystal violet stain solution, drained of excess dye, and applied to the treated areas for 5 minutes. The discs were then removed and the excess dye washed off by several water rinses. The resulting stained skin areas were then photographed. A typical result is shown in FIG. 3 below. Clearly, both Vaseline and Formulation 1 (DermSeal) were effective.

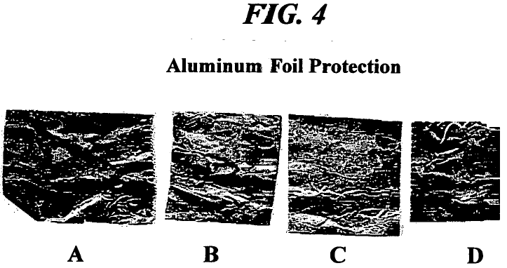

The skin-protecting effect of Formulation 1 was also demonstrated by conducting an modification of the aluminum foil deterioration test. In this assay, pieces of aluminum foil are first coated with 50 microliters of the test gel and air dried for 10 minutes, The coated foil area is then exposed to 100 microliters of 3N HCl acid for 30 minutes. The results of one such test is presented in FIG. 4.

The control (A) piece of foil developed a small hole. By contrast, a variant of Formulation 1 (C), composed of 4% DRY-FlO/AF modified starch, 8% Mineral Oil and 1% Benzalkonium Chloride, and Formulation 5 , an emulsion composed of 4% natural starch, 8% Petrolatum, 5% Mineral Oil, 1% Polysiloxane, 4% Dimethyl cyclopentasiloxane, 10% glycerol, and 0.5% Benzalkonium Chloride, did not develop any holes. By contrast, Petrolatum alone when applied to aluminum foil did not prevent the development of holes (B).

### Example 3

### Reversible Hydration Effects of Topically Applied Thixogels

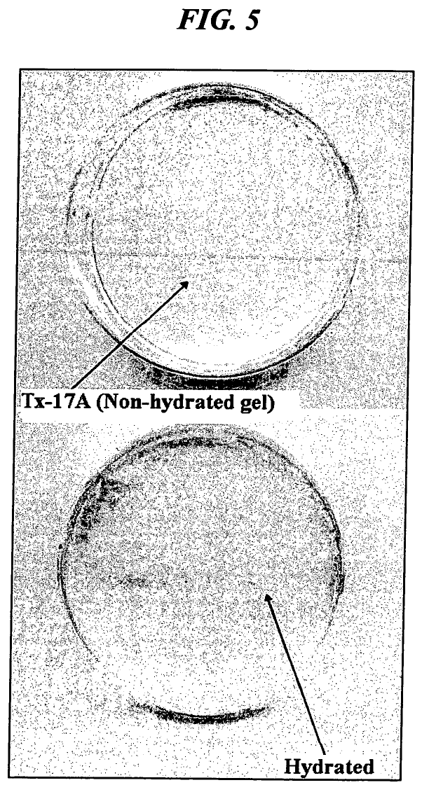

A remarkable property of all Thixogel formulations is their ability to be air-dried and then to rehydrate back to their original volume, upon addition of water. This is seen for a sample of Formulation 5 as shown in FIG. 5.

This phenomenon occurs when the gel is applied to skin. After drying, it can be rehydrated with water, and this can be repeated through many cycles of drying and rehydration. Moreover, upon drying on the hands, they may be rinsed in 95% ethanol and air-dried without preventing rehydration upon subsequent exposure to water. This unique property we have called, a “glove in a glove.” It may have wide ranging benefits for healthcare workers who get dry irritated skin because they repeatedly wash their hand multiple times a day often employing intervening alcohol washes.

### Example 4

### Delivery of Oxygen from a Thixogel Formulation

It was speculated that starch-coated oil droplets might bind and then slowly release dissolved oxygen. Oxygen may be incorporated in such systems by using Perfluorodecalin as an oil. This material is widely used to bind oxygen and as a blood substitute. It has been incorporated into emulsions in a number of patents ^{50 56}(U.S. Pat. Nos. 4,569,784; 3,277,013; 3,778,381; 3,823,091; 3,993,581).

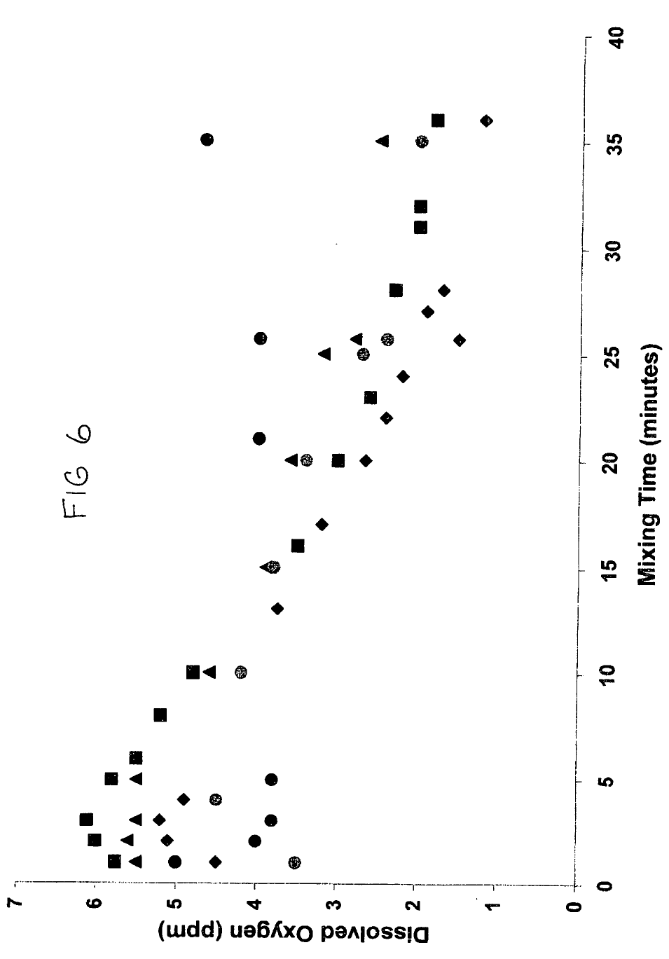

Dissolved oxygen was incorporated into a Thixosol formulation 6 by replacing all other oil phase ingredients with 10% Perfluorodecalin (PFC). This formulation is called OxyTega. In order to achieve this effect, various aqueous solutions were oxygen charged. These were composed of just one added component of OxyTega gel or OxyTega gel, itself. Oxygen was bubbled directly into the solution for 5 minutes at 20 psi in an open-air container. The oxygenated solutions obtained were then continuously stirred at 25° C., at moderate speed and dissolved oxygen was continuously monitored with an oxygen electrode connected to an oxygen meter. The results are summarized in FIG. 6.

The kinetic curves for all Thixogel components, with or without Perfluorodecalin share a similar oxygen release rate and have an approx. half-life of 15 minutes. By contrast, OxyTega based systems retain the dissolved oxygen over the 30 minutes. There is, in fact, a trend toward increasing the amount of oxygen available for release beyond 30 minutes. Similar tests conducted on Thixosol emulsions employing Mineral oil and 1% Benzalkonium chloride show a half-life of approximately 90 minutes. The most favorable starch/mineral oil ratio for achieving slow oxygen release occurred at 1:3 ratio.

### Example 5

### Anti-Microbial Thixogel Formulations

Benzalkonium Chloride, at 0.5%, acts as both a surfactant and anti-bacterial in Thixogel formulations. Given concerns about possible skin irritation at or above 0.5%, the concentration was reduced to 0.1%. Palmitoleic Acid was also added to supplement the emulsifying action and, at the same time, to increase the overall anti-microbial action of the Benzalkonium Chloride/Palmitoleic Acid combination.

SanoSeal Gel (Formulation 8) was tested for its bactericidal action on a clinical isolate of Staphylococcus aureus. The bacteria were applied at a level of 5×10⁵ cells to a saline moistened sterile filter paper and exposed for 20 minutes to Formulation 8 (100 μL per filter) to completely cover the bacterized paper.

Controls included sterile filter papers with an equal number of bacteria, These were covered with a sterile starch/oil dispersion lacking Palmitoleic acid (positive control). Sterile filter papers, with no bacteria and covered with sterile Formulation 8 were also employed as controls. After treatment, the filter papers were aseptically transferred to a sterile broth and incubated on a rotary shaker overnight. It was found that bacterized paper without Palmitoleic acid in Thixogel was clouded by growth of bacteria. By contrast, filter papers either without bacteria or coated with SanoGel were as clear as uninoculated sterile broth. Small aliquots from each broth were then transferred to a fresh sterile broth and incubated again overnight at 37° C. Only the cloudy broth from the bacterized Thixogel-treated flask grew out bacteria. These results show that Formulation 8 (SanoSeal Gel) kills up to five-logs of applied bacteria in a 20-minute exposure. Since the formulation contains no toxic chemicals, and no drying alcohol, it is effective and safe and is also not harsh or irritating to skin.

### Example 6

### Novel Delivery System for Hydrophobic Plant Actives

Once desired plant actives have been selected for the intended formulation a proper vehicle for their delivery must be designed. Formulation 5 (EktaDerm) was chosen as the best delivery system for hydrophobic plant actives for the following reasons: 1) hydrophobic plant active compounds are soluble in oil phase ingredients, 2) dry powders can be prepared by exhaustive venting of volatile solvents, 3) dry powders of hydrophobic plant active compounds are soluble in mineral oil, and pant actives dissolved in mineral oil are also soluble in combined oil phase ingredients of EktaDerm(formulation 5).

In addition, protection of plant anti-oxidants from light and air can be achieved by adding Tocopherol (Vitamin E) directly to the mineral oil prior to dissolving the plant active.

We have incorporated several different hydroalcoholic extracts of plants rich in flavonoids. For example, green onion leaves are a rich source of the flavonoid, Quercetin. A hydroalcoholic extract of green onion leaves , here designated “Allin,” was prepared and tested for its effect on the proliferation of normal human keratinocytes cultured in a serum-free medium containing insulin and retinyl acetate. Both of these are the sole growth factors required for autocrine growth of keratinocytes (Wille, 2003). Our results (FIG. 6) showed that Allin inhibited autocrine growth of proliferating keratinocytes, and the effect was equivalent to 10 μM Quercetin dihydrate. These results indicate that green onion leaf containing flavonoids, like Quercetin, block autocrine growth of serum-free cultures of keratinocytes by inhibiting the mitogenic signal transduction cascade. This action occurs through the insulin-like growth factor receptor (IGFR), which is itself activated by retinoid stimulation and inhibited by inhibitors of tyrosine protein kinases.

### Example 7

### Antioxidant Plant Extracts

We have discovered several good anti-oxidant plant extracts as candidates for incorporation into our chosen Thixogel hydrophobic delivery system (Formulation 5, EktaDerm). Moreover, it is our claim that plant extracts with strong antioxidant activity will be useful sources of plant-derived anti-irritants.

The anti-oxidant activity of anti-irritant plant extracts was assayed by the diphenylpicrylhydrazyl radical (DPPH*) test (Bonina et al, 2002). Table 2 summarizes these results.

TABLE 2
Relative Antioxidant Activity of Plant Extracts
(Values from DPPH* Assay)

| | Relative Antioxidant | Activity Relative to |
|---|---|---|
| | Potency, P = (EC₅₀ × | Vitamin E (EC₅₀ |
| Plant Extract (% Ethanol) | Conc. Factor, Kg/L) | of 46 μM = 1 unit |
| Indole Acetic Acid (0) | 2.0 | 4.1 |
| Green Onion Leaf (50) | 3.1 | 6.7 |
| Red Swiss Chard (50) | 4.0 | 5.2 |
| Tomato Paste (50) | 13.3 | 1.6 |
| Corn Tassel (95) | 16.5 | 1.3 |
| Autumn Olive Berry (50) | 176 | 0.1 |

In our search for a good plant-derived anti-oxidant, Autumn Olive (Elaeagnus umbellata) was found to be a very rich source of anti-oxidants, as were Cranberry juice, and grapefruit seed oil(CITRICIDAL). Two other sources of anti-oxidants were found to be hydroalcoholic extracts of corn tassels (Tasselin) and tomato paste. In addition, we have also isolated lycopenes from both tomato paste and Autumn Olive berries. They are both rich sources of carotenes, and have been incorporated into Formulations 10 and 11(PhytoSeal Gels). Similarly, hydroalcoholic extracts of green onion leaves and red Swiss chard have demonstrated modest but significant anti-oxidant activity. Green onion leaf extract was incorporated into Formulation 12 along with Retinyl Acetate to enhance the anti-oxidant properties of this formulation.

In summary, we have described a process for producing stable dispersions of oil droplets in a starch matrix. The process first requires gelatinizing natural starch at a temperature sufficient to dissolve starch in an aqueous solution containing one or more emulsifying agents and, then blending of one or more oils with the gelatinized starch phase at a temperature sufficient to prevent gel formation.

The use of petrolatum, mineral oil and silicon oils, in various combinations, in the oil phase ingredients, is employed in specified amounts, to produce a greaseless and tackless gel having good spreadability, rapid drying, and water-resistance. These materials form a protective film on skin. Glycerol, a humectant, can be used in the aqueous phase to provide for skin moisturization.

Hydrocolloid gels prepared by the above process are capable of topically delivering plant-derived hydrophobic extracts such as cosmetic and medicinal ingredients into the oil-phase ingredients of the starch-in-oil gel systems These hydrocolloid gel emulsion confer anti-oxidant, anti-aging, and anti-irritant properties as obtained with the formulations set forth at the end of the chapter. In conclusion Formulation 5 (EktaDerm) Thixogel system is a novel topical delivery system with diverse personal care applications.

It should be readily apparent that Thixogel emulsions are both easy to formulate and cost-effective. The major ingredients such as cornstarch, mineral oil, and petrolatum are relatively inexpensive. Since a stable hydrogel emulsion of starch-in-oil requires only very low levels of an emulsifying agent, the formulator can avoid the use of expensive fatty acid alcohols, fatty acid esters, thickeners, and emulsion stabilizers, that are generally required to produce stable oil-in-water emulsions.

Unlike many cosmetic emulsions, Thixogel emulsions are completely greaseless, and leave no oily residue on the skin. Furthermore, they are completely resistant to alcohol and thus do not wash off when body skin is rinsed or decontaminated with alcohol. This property makes them highly useful to healthcare workers, who can avoid the irritant effects of multiple cycles of water and alcohol washes during the course of their sanitary protocols.

Finally, Thixogel formulations lend themselves to the formulation of oil-soluble active ingredients. Plant extracts that are only soluble in organic solvents can be readily dried down, re-dissolved in mineral oil, and employed with the oil phase chosen. In this regard, it has been it has been shown that lycopenes, derived from tomato paste, and carotenes, derived from carrots can be prepared in mineral oil and added directly to the oil phase ingredients during formulation of EktaDerm type gels.

### Example 8

### List of Formulations

Formulation 1
DermSeal - Basic Skin Barrier Gel: Hydrophobic Topical Gel
Delivery System

| Ingredient | | Function(s) | Wt. % |
|---|---|---|---|
| A. | Petrolatum jelly | Oleophilic phase | 7.5 |
| | | (skin protectant) | |
| B. | Deionized Water | Water phase (hydration) | 88.0 |
| | Corn Starch | Thickener, film-forming gel | |
| | Benzalkonium Chloride | Emulsifier(preservative) | 0.5 |
| | CITRICIDAL | Natural Preservative | 0.5 |

Weigh the Part A ingredient and heat at 50° C. until thoroughly melted in a suitable vessel equipped with a mixer Add C ingredient to pre-heated Part A ingredient. Weigh the Part B starch ingredient, and place in a suitable vessel equipped with low-shear mixer. Add a sufficient volume of deionized water to produce a 0.5% concentration of benzalkonium chloride. Heat the Part B ingredients at 90° C. until the starch is entirely dissolved. Remove from heat, add directly to heated Part A ingredient and then heat at 65° C. with continuous mixing until a homogeneous emulsion is formed. Tip: Keep the gelated starch above its boiling point during the petrolatum jelly blending step. Store the gel in sealed container away from heat and air. # The ratio of starch to petrolatum can be varied from 1:1 to 1:3 to 3:1 and still result in a stable homogenous emulsified gel. A variety of pure food grade (pfg) corn starches have been tried in this type of formulations. These include Staley, Pure Food Powder, Decatur, Ill., and DRY-FLO® AF, a modified starch from National Starch & Chemical, Co. Mineral oil can be substituted for Petrolatum Jelly. A non-ionic surfactant, e.g., Triton-X100 (0.1 Wt. %) or an anionic surfactant, e.g., Sodium Lauryl Sulfate (0.5 Wt %) can be substituted for Benzalkonium Chloride.

Formulation 2
EktaSeal - Skin Barrier and Moisturizing Gel: Hydrophobic
Topical Delivery - System

| Ingredient | | Function(s) | Wt. % |
|---|---|---|---|
| A | Petrolatum jelly#@ | Oleophilic phase | 8.8 |
| | | (skin protectant) | |
| B | Deionized Water | Water phase (hydration) | 73.0 |
| | Corn Starch (pfg)*# | Thickener | |
| | | (gel and film-forming agent) | 3.7 |
| | Benzalkonium Chloride | Emulsifier(preservative) | 1.0 |
| | Glycerol | Humectant | 20.0 |
| C. | CITRICIDAL | Natural preservative | 0.5 |

Weigh the Part A ingredient and heat at 50° C. until thoroughly melted in a suitable vessel equipped with a mixer and add Part C ingredient. Weigh the Part B starch ingredient and place in suitable vessel equipped with mixer. Add a sufficient volume of deionized water, glycerol, and benzalkonium chloride, and heat the Part B ingredients at 90° C. until the starch is entirely dissolved. Remove from heat and add to pre-heated Part A and Part C ingredients. Heat at 65° C. with continuous mixing until a homogeneous emulsion is formed. Keep the gelated starch above its boiling point during the petrolatum blending step. Store the gel in sealed container away from heat and air.

The ratio of starch to petrolatum can be varied from 1:1 to 1:3 to 3:1 and will still result in a stable homogenous emulsified gel. A variety of pure food grade (FFG) corn starches can be used including Stanley, Pure Food Powder, National Starch & Chemical Co., and Argo. Mineral Oil, USP may be substituted for Petrolatum Jelly.

Formulation 3
VegaSeal - All Natural Skin Moisturizing Gel: Hydrophobic
Topical Delivery System

| Ingredient | | Function(s) | Wt. % |
|---|---|---|---|
| A | Soy Bean Oil | Oleophilic phase- | 3.5 |
| | | (skin softener/anti-irritant) | |
| B | Deionized Water | Water phase (Hydration) | 82.0 |
| | Corn Starch | Thickener, oil absorber | 3.0 |
| | (StaMist 365) | | |
| | Lecithin | Vegetable Emulsifier | 1.0 |
| | Glycerol | Humectant | 10 |
| C. | CITRICIDAL | Natural Preservative | 0.5 |

Weigh the Part B cornstarch ingredient and place in suitable vessel equipped with a mixer Add a sufficient volume of deionized water and glycerol. Heat these Part B ingredients at 90° C. until the starch is entirely dissolved. Remove from heat and add the remainder of Part B ingredient (Lecithin in 5% Ethanol). Mix the combined Part B ingredients with Part A (Soy Bean Oil) and Part C ingredients. Heat the combined oil-water mixture at 65° C. with continuous mixing until a homogeneous emulsion is formed. Keep the gelated starch above its boiling point during the Soy Bean Oil blending step. Store the gel in sealed container away from heat and air. Sta-Mist 365, is a hydrophobically-modified corn starch from Stanley Manufacturing Co., Decatur, Ill. Meadowfoam Oil, Oleic Acid, Olive Oil and Canola Oil can be substituted for Soy Bean Oil.

Formulation 4.
SilkDerm - Moisturizing Skin Barrier Gel: Topical Delivery
System

| Ingredient | | Function(s) | Wt. % |
|---|---|---|---|
| A | Dimethicone (200 ® Fluid) | Oleophilic skin protectant | 5.6 |
| B | Deionized Water | Water phase (hydration) | 79.5 |
| | Corn Starch (pfg) | Thickener | 3.4 |
| | | (gel and film-forming agent) | |
| | Benzalkonium Chloride | Emulsifier | 1.0 |
| | Glycerol | Humectant | 10.0 |
| C. | CITRICIDAL | Natural preservative | 0.5 |

Weigh the Part B starch ingredient and place in suitable vessel equipped with mixer. Add sufficient volume of deionized water, glycerol and benzalkonium chloride, mix thoroughly, and heat the Part B ingredients at 90° C. until the starch is entirely dissolved. Remove from heat and add Citricidal to Part A ingredient (Dimethicone). Heat at 65° C. with continuous mixing until a homogeneous emulsion is formed. Keep the gelated starch above its boiling point during the Dimethicone blending step. Store the gel in sealed container away from heat and air. Dimethicone (dimethyl polysiloxane, 60,000 cps). A variety of pure food grade (pfg) corn starches can be employed including those from Stanley, National Starch, and Argo.

Formulation 5
EktaDerm - Basic Topical Delivery System for Hydrophobic
Plant Actives

| Ingredient | | Function(s) | Wt. % |
|---|---|---|---|
| A | Poly(dimethylsiloxane) | Skin protectant | 0.8 |
| | (DC-200 Fluid ®) | | |
| | Decamethyl | Skin protectant | 3.3 |
| | Cyclopentasiloxanes | | |
| | (DC-245 Fluid ®) | | |
| | Mineral Oil | Oleophilic phase | 4.1 |
| | Petrolatum Jelly | Oleophilic phase | 9.3 |
| B | Deionized Water | Water phase (hydration) | 68.1 |
| | Corn Starch (pfg) | Thickener | 3.1 |
| | | (gel and film-forming | |
| | | agent) | |
| | Benzalkonium Chloride | Emulsifier | 0.8 |
| | Glycerol | Humectant | 10.0 |
| C. | CITRICIDAL | Natural preservative | 0.5 |

Weigh the Part B starch ingredient and place in suitable vessel equipped with mixer. Add a sufficient volume of deionized water, glycerol and benzalkonium chloride, mix thoroughly, and heat the Part B ingredients at 90° C. until the starch is entirely dissolved. Remove from heat and cool to 65° C. Weight out Part A ingredient (mineral oil, DC-200, DC-245 and petrolatum jelly) and add directly to pre-heated Part B ingredients. Stir in Part C ingredient, and mix continuously until a homogeneous emulsion is formed. Keep the gelated starch above its boiling point during the addition of Part A ingredients. Store the gel in sealed container away from heat and air. A variety of pure food grade (pfg) corn starches can be employed including Stanley, National Starch, and Argo.

Formulation 6
OxyTega Gel - Skin Barrier Topical Delivery System

| Ingredient | | Function(s) | Wt. % |
|---|---|---|---|
| A | Perflurodecalin$ | Oleophilic phase | 9.5 |
| | | (Oxygen binding agent) | |
| B | Deionized Water | Water phase (Hydration) | 84.5 |
| | Corn Starch (pfg) | Thickener | 4.0 |
| | | (gel and film-forming agent) | |
| | Benzalkonium Chloride | Emulsifier | 1.5 |
| C. | CITRICIDAL | Natural preservative | 0.5 |

Weigh the Part B starch ingredient and place in suitable vessel equipped with mixer. Add a sufficient volume of deionized water, and add the benzalkonium chloride. Mix thoroughly, and heat the Part B ingredients at 90° C. until the starch is entirely dissolved. Remove from heat and add Part C ingredient to Part A ingredient (Perfluorodecalin) and heat at 65° C. with continuous mixing until a homogeneous emulsion is formed. Keep the gelated starch above its boiling point during the Perfluorodecalin blending step. Store the gel in sealed container away from heat and air. A ‘variety of pure food grade (pfg) corn starches can be employed including those from Stanley, National Starch, and Argo. Perfluorodecalin (95%, Aldrich Company, Milwaukee, Wis. 53201).

Formulation 7
Itch-Relief Gel - Skin Barrier: Witch Hazel Delivery System

| Ingredient | | Function(s) | Wt. % |
|---|---|---|---|
| A | Petrolatum Jelly | Oleophilic phase | 8.0 |
| | | (skin protectant) | |
| | Poly(dimethylsiloxanes) | Skin protectant | 1.0 |
| B | Potato Starch | Rheology modifier | 4.0 |
| | (Structure ® Solance) | | |
| | Benzalkonium Chloride | Emulsifier | 1.0 |
| | Hammelis Water | Water phase | 71.5 |
| | (86%, Witch Hazel) | (astringent) | |
| | Isopropyl Alcohol | skin disinfectant | 14.0 |
| C. | CITRICIDAL | Natural preservative | 0.5 |

Weigh the Part B starch ingredient and place in suitable vessel equipped with mixer. Add a sufficient volume of Hammelis Water (14% isopropyl alcohol), and benzalkonium chloride. Mix thoroughly and heat the Part B ingredients at 90° C. until the starch is entirely dissolved. Remove from heat and add Part C ingredient to Part A ingredient (Petrolatum jelly) and heat at 65° C. with continuous mixing until a homogeneous—emulsion is formed. Keep the gelated starch above its boiling point during the petrolatum and Simethicone blending step. Store the gel in sealed container away from heat and air. Structure Solance is 2-(N,N-bis(2-carboxyethylamino)ethyl ether -derivatized potato starch from National Starch & Chemical, Bridgewater, N.J. Witch Hazel, U.S.P. * 200 Fluid from Dow Corning, Midland, Mich.

Formulation 8.
SanoSeal Gel - Antimicrobial Hand Lotion: Hydrophobic
Plant Active Delivery System

| Ingredient | | Function(s) | Wt. % |
|---|---|---|---|
| A | Petrolatum Jelly | Oleophilic phase | 6.6 |
| | | (skin protectant) | |
| | Poly(dimethylsiloxanes) | Skin protectant | 5.0 |
| | Palmitoleic Acid | Anti-microbial agent | 0.5 |
| B | Deionized Water | Water phase (hydration) | 84.0 |
| | Corn Starch | Rheology modifier | 3.3 |
| | (DRY-FLO ® AF) | | |
| | Benzalkonium Chloride | Emulsifier | 0.1 |
| C. | CITRICIDAL | Natural preservative | 0.5 |

Weigh the Part B starch ingredient and place in suitable vessel equipped with mixer. Add a sufficient volume of deionized water and benzalkonium chloride, mix thoroughly, and heat the Part B ingredients at 90° C. until the starch is entirely dissolved. Remove from heat and add Part C ingredient to Part A ingredient (Petrolatum Jelly, Simethicone, and Palmitoleic Acid), and heat at 65° C. with continuous mixing until a homogeneous emulsion is formed. Keep the gelated starch above its boiling point during the petrolatum and Simethicone blending step. Store the gel in sealed container away from heat and air.

DRY-FLO® AF (28-1805/KAX-3052) is the calcium salt of amylopectin starch, modified chemically with adduct, dodecenylbutanedioate, from National Starch & Chemical Co., Bridgewater, N.J. Palmitoleic Acid provides antibacterial activity (see Reference, page). 200 Fluid from Dow Corning, Midland, Mich. Oleic Acid (0.5%) can be substituted for Palmitoleic Acid.

Formulation 9
PhytoSeal L - Anti-Irritant Plant Active Topical Delivery System

| Ingredient | | Function(s) | Wt. % |
|---|---|---|---|
| A | Petrolatum Jelly | Oleophilic phase | 9.3 |
| | | (skin protectant) | |
| | Poly(dimethylsiloxanes) | Skin protectant | 0.8 |
| | Decamethyl cyclosiloxanes | Skin protectant | 3.2 |
| | Mineral Oil | Oleophilic solvent | 4.0 |
| B | Deionized Water | Water phase (hydration) | 68.0 |
| | Corn Starch (pfg)# | Rheology modifier | 3.2 |
| | | (film and gel forming agent) | |
| | Benzalkonium Chloride | Emulsifier | 0.8 |
| | Glycerol | Humectant | 10.0 |
| C. | Tomato paste extract | Plant active | 0.1 |
| | | (anti-aging agent) | |
| | CITRICIDAL | Natural preservative | 0.5 |
| | | And antioxidant | |
| | Tocopherol | Anti-oxidant | 0.1 |

Weigh the Part B starch ingredient and place in suitable vessel equipped with mixer. Add a sufficient volume of deionized water, glycerol and benzalkonium chloride, mix thoroughly, and heat the Part B ingredients at 90° C. until the starch is entirely dissolved. Remove from heat and add Part D ingredient (Tocopherol and CITRICIDAL) to Part A ingredient (Petrolatum Jelly, 200® Fluid, 245® Fluid). Part C ingredient (lycopene solution in mineral oil) is added to Part A ingredients and heated at 65° C. and then added to Part B ingredients with continuous mixing until a homogeneous emulsion is formed. Keep the gelated starch above its boiling point during the Part A ingredients blending step. Store the gel in sealed container away from light, heat and air. Corn starch from Staley, Decatur, Ill. 200® Fluid and 245® Fluid from Dow Corning, Midland, Mich. A Lycopene-enriched extract was purified from tomato paste and a saturated solution of it dissolved in Mineral Oil, USP.

Formulation 10
PhytoSeal C/L - Photo-aged Skin Repair Gel: Hydrophobic
Plant Active Delivery System

| Ingredient | | Function(s) | Wt. % |
|---|---|---|---|
| A. | Petrolatum Jelly | Oleophilic phase | 9.2 |
| | | (skin protectant) | |
| | Poly(dimethylsiloxanes) | Skin protectant | 0.8 |
| | Decamethyl cyclosiloxanes | Skin protectant | 3.2 |
| | Sea Buckthorn Oil | Oleophilic solvent | 4.0 |
| B. | Deionized Water | Water phase (hydration) | 68.0 |
| | Corn Starch (pfg) | Rheology modifier | 3.2 |
| | | (film and gel forming agent) | |
| | Benzalkonium Chloride | Emulsifier | 0.8 |
| | Glycerol | Humectant | 10.0 |
| C. | Carrot Extract | Plant active | 0.1 |
| | | (Anti-oxidant) | |
| | Tomato Paste Extract | Plant active | 0.1 |
| | | (Anti-oxidant) | |
| D. | Citricidal | Natural preservative | 0.5 |
| E. | Tocopherol | Anti-oxidant | 0.1 |

Weigh the Part B starch ingredient and place in suitable vessel equipped with mixer. Add a sufficient volume of deionized water, glycerol and benzalkonium chloride, mix thoroughly, and heat the Part B ingredients at 90° C. until the starch is entirely dissolved. Remove from heat. Add Part D and E ingredients (Tocopherol and Citricidal) to Part A ingredients (Petrolatum Jelly, 200® Fluid, 245® Fluid, Mineral Oil) at 65° C. and mix Part B ingredients with continuous stirring until a homogeneous emulsion is formed. Part C ingredients (Carrot and Tomato Paste Extracts) in mineral were added to Part A ingredients prior to mixing with Part B ingredients at 65° C. until thorough mixing is achieved. Keep the gelated starch above its boiling point during the petrolatum, mineral oil and silicon oil-blending step. Store the gel in a sealed amber container away from light, heat and air. Corn starch from Staley, Decatur, Ill. 200® Fluid and 245® Fluid from Dow Corning, Midland, Mich. Carotene-enriched and lycopene-enriched preparations were prepared as saturated solutions in mineral oil.

Formulation 11
PhytoSeal T - Anti-aging: Plant Active Topical Delivery System

| Ingredient | | Function(s) | Wt. % |
|---|---|---|---|
| A. | Petrolatum Jelly | Oleophilic phase | 9.2 |
| | | (skin protectant) | |
| | Poly(dimethylsiloxanes) | Skin protectant | 0.8 |
| | Decamethyl cyclosiloxanes | Skin protectant | 3.2 |
| | Mineral Oil | Oleophilic solvent | 4.0 |
| B. | Deionized Water | Water phase (hydration) | 68.0 |
| | Corn Starch | Rheology modifier | 3.2 |
| | | (film and gel forming agent) | |
| | Benzalkonium Chloride | Emulsifier | 0.8 |
| | Glycerol | Humectant | 10.0 |
| C. | Corn Tassel Extract | Plant Active | 0.1 |
| | | (Anti-irritant) | |
| | Retinol | Anti-aging agent | 0.1 |
| D. | CITRICIDAL | Natural preservative | 0.5 |
| E. | Tocopherol | Anti-oxidant | 0.1 |

Weigh the Part B starch ingredient and place in suitable vessel equipped with mixer. Add a sufficient volume of deionized water, glycerol and benzalkonium chloride, mix thoroughly, and heat the Part B ingredients at 90° C. until the starch is entirely dissolved. Remove from heat. Add Part D ingredient (Tocopherol and Citricidal) to Part A ingredients (Petrolatum Jelly, 200® Fluid, 245® Fluid, Mineral Oil) at 65° C. and mix Part B ingredients with continuous stirring until a homogeneous emulsion is formed. Part C ingredients (Tasselin and Retinol) were prepared separately as solutions in 95% ethanol and added to the combined Part A, Part B and Part D mixture at 65° C. until thorough mixing is achieved. Tip: Keep the gelated starch above its boiling point during the petrolatum, mineral oil and silicon oil-blending step. Store the gel in a sealed amber container away from light, heat and air. Corn starch from Staley, Decatur, Ill. 200® Fluid and 245® Fluid from Dow Corning, Midland, Mich. Tasselin™, a phenoxyacetic acid ester-enriched from hydroalcoholic extract of tassels from corn plants, was dissolved in 95% ethanol.

Formulation 12
PhytoSeal R/O - Anti-Wrinkling: Plant Active Topical Delivery
System

| Ingredient | | Function(s) | Wt. % |
|---|---|---|---|
| A. | Petrolatum Jelly | Oleophilic phase | 9.2 |
| | | skin protectant) | |
| | Poly(dimethylsiloxanes) | Skin protectant | 0.8 |
| | Decamethyl cyclosiloxanes | Skin protectant | 3.2 |
| | Mineral Oil | Oleophilic solvent | 4.0 |
| B. | Deionized Water | Water phase (hydration) | 68.0 |
| | Corn Starch (pfg) | Rheology modifier | 3.2 |
| | | (film and gel forming agent) | |
| | Benzalkonium Chloride | Emulsifier | 0.8 |
| | Glycerol | Humectant | 10.0 |
| C. | Retinyl Acetate | Anti-wrinkling agent | 0.1 |
| | Onion Leaf Extract | Plant Active | 0.1 |
| | | (Anti-oxidant) | |
| D. | CITRICIDAL | Natural preservative | 0.5 |
| E. | Tocopherol | Anti-oxidant | 0.1 |

Weigh the Part B starch ingredient and place in suitable vessel equipped with mixer. Add a sufficient volume of deionized water, glycerol and benzalkonium chloride, mix thoroughly, and heat the Part B ingredients at 90° C. until the starch is entirely dissolved. Remove from heat. Add Part D and E ingredients (Tocopherol and Citricidal) to Part A ingredients (Petrolatum Jelly, 200® Fluid, 245® Fluid, Mineral Oil) at 65° C. and mix Part B ingredients with continuous stirring until a homogeneous emulsion is formed. Part C ingredients (Retinyl Acetate and Onion Leaf Extract) were prepared separately as 1 % solutions in 95% ethanol and added to the combined Part A, Part B and Part D mixture at 65° C. until thorough mixing is achieved. Keep the gelated starch above its boiling point during the petrolatum, mineral oil and silicon oil-blending step. Store the gel in a sealed amber container away from light, heat and air. Corn starch from Staley, Decatur, Ill. 200® Fluid and 245™ Fluid from Dow Corning, Midland, Mich. A Quercetin-enriched extract was purified from Onion Leaf Extract and dissolved in 95% ethanol.

Formulation 13
UltraDerm-Plus - Topical Delivery System

| Ingredient | | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 0.33 |
| | DC-245 Fluid ® | | |
| | Mineral Oil | Oleophilic phase | 4.1 |
| | Petrolatum Jelly | Oleophilic phase | 9.3 |
| B | Deionized Water | Water phase (hydration) | 68.0 |
| | Corn Starch | Thickener (gel/ | 3.1 |
| | | film-forming agent) | |
| | Benzalkonium Chloride | Emulsifier | 0.8 |
| | Glycerol | Humectant | 10.0 |
| | Poly(oxy)ethylene-WSR | Lubricity/slip | 0.1 |
| C. | CITRICIDAL | Natural preservative | 0.5 |

### Procedure

Weigh the Part B starch ingredient and place in suitable vessel equipped with mixer. Add a sufficient volume of deionized water, glycerol and benzalkonium chloride, and polyethylene oxide(POLYOX), mix thoroughly, and heat the Part B ingredients at 90° C. until the starch is entirely dissolved. Remove from heat and cool to 65° C. Weight out Part A ingredient (mineral oil, DC-200, DC-245 and petrolatum jelly) and add directly to pre-heated Part B ingredients. Stir in Part C ingredient, and mix continuously until a homogeneous emulsion is formed. Keep the gelated starch above its boiling point during the addition of Part A ingredients. Store the gel in sealed container away from heat and air. A variety of pure food grade cornstarches can be employed including Stanley, National Starch, and Argo.

Formulation 14
Berri-Derm: A Natural Emollient Topical Delivery System

| Ingredient | | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 0.33 |
| | DC-245 Fluid ® | | |
| | Mineral Oil | Oleophilic phase | 4.1 |
| | Berry Wax | Oleophilic phase | 9.3 |
| | EnviroPure310 ® | | |
| B | Deionized Water | Water phase (hydration) | 68.1 |
| | Corn Starch | Thickener (gel/ | 3.1 |
| | | film-forming agent) | |
| | Benzalkonium Chloride | Emulsifier | 0.8 |
| | Glycerol | Humectant | 10.0 |
| C. | CITRICIDAL | Natural preservative | 0.5 |

### Procedure

Weigh the Part B starch ingredient and place in suitable vessel equipped with mixer. Add a sufficient volume of deionized water, glycerol and benzalkonium chloride, mix thoroughly, and heat the Part B ingredients at 90° C. until the starch is entirely dissolved. Remove from heat and cool to 65° C. Weight out Part A ingredient (mineral oil, DC-200, DC-245 and Berry Wax/Olive Oil, EnviroPure310, React-NTI) and add directly to pre-heated Part B ingredients. Stir in Part C ingredient, and mix continuously until a homogeneous emulsion is formed. Keep the gelated starch above its boiling point during the addition of Part A ingredients. Store the gel in sealed container away from heat and air. A variety of pure food grade cornstarches can be employed including Stanley, National Starch, and Argo.

Formulation 15
PolyDerm - Topical Gel Delivery System

| Ingredient | | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 0.33 |
| | DC-245 Fluid ® | | |
| | Mineral Oil | Oleophilic phase | 4.1 |
| | Petrolatum Jelly | Oleophilic phase | 7.5 |
| B | Deionized Water | Water phase (hydration) | 75.0 |
| | Guar Gum | Gel Thickener | 1.0 |
| | Benzalkonium Chloride | Emulsifier | 0.8 |
| | Glycerol | Humectant | 10.0 |
| C. | CITRICIDAL | Natural preservative | 0.5 |

### Procedure

Weigh the Part B guar gum ingredient and place in suitable vessel equipped with mixer. Add a sufficient volume of deionized water, glycerol and benzalkonium chloride, mix thoroughly, and heat the Part B ingredients at 90° C. until the gum is entirely dissolved. Remove from heat and cool to 65° C. Weight out Part A ingredient (mineral oil, DC-200, DC-245 and petrolatum jelly) and add directly to pre-heated Part B ingredients. Stir in Part C ingredient, and mix continuously until a homogeneous emulsion is formed. Tip: Keep the gum above its melting point during the addition of Part A ingredients. Store the gel in sealed container away from heat and air.

Formulation 16
SynDerm - Basic Topical Delivery System for Hydrophobic
Plant Actives

| Ingredient | | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 0.33 |
| | DC-245 Fluid ® | | |
| | Mineral Oil | Oleophilic phase | 4.1 |
| | Petrolatum Jelly | Oleophilic phase | 9.3 |
| B | Deionized Water | Water phase (hydration) | 72.2 |
| | Carboxymethylcellulose | Thickener (gelforming agent) | 2.0 |
| | Benzalkonium Chloride | Emulsifier | 0.8 |
| | Glycerol | Humectant | 10.0 |
| C. | CITRICIDAL | Natural preservative | 0.5 |

### Procedure

Weigh the Part B Carboxymethylcellulose, sodium salt(CMC) ingredient and place in suitable vessel equipped with mixer. Add a sufficient volume of deionized water, glycerol and benzalkonium chloride, mix thoroughly, and heat the Part B ingredients at 90° C. until the CMC is entirely dissolved. Remove from heat and cool to 65° C. Weight out Part A ingredient (mineral oil, DC-200, DC-245 and petrolatum jelly) and add directly to pre-heated Part B ingredients. Stir in Part C ingredient, and mix continuously until a homogeneous emulsion is formed. Keep the CMC above its boiling point during the addition of Part A ingredients. Store the gel in sealed container away from heat and air.

Formulation 17
PolycelluDerm - A Microcrystalline Cellulose Gel Topical
Delivery System

| Ingredient | | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 0.33 |
| | DC-245 Fluid ® | | |
| | Mineral Oil | Oleophilic phase | 4.1 |
| | Petrolatum Jelly | Oleophilic phase | 9.3 |
| B | Deionized Water | Water phase (hydration) | 72.2 |
| | Guar gum | Gel Thickener | 0.5 |
| | Cellulose/cellulose gum | Rheological modifier | 1.5 |
| | AVICEL 611(FMC) | | |
| | Benzalkonium Chloride | Emulsifier | 0.8 |
| | Glycerol | Humectant | 10.0 |
| C. | CITRICIDAL | Natural preservative | 0.5 |

### Procedure

Weigh the Part B microcrystalline cellulose/cellulose gum (AVICEL 611, FMC) ingredient and place in suitable vessel equipped with mixer. Add a sufficient volume of deionized water, glycerol and benzalkonium chloride, mix thoroughly, and heat the Part B ingredients at 90° C. until the gum is entirely dissolved. Remove from heat and cool to 65° C. Weight out Part A ingredient (mineral oil, DC-200, DC-245 and petrolatum jelly) and add directly to pre-heated Part B ingredients. Stir in Part C ingredient, and mix continuously until a homogeneous emulsion is formed. Keep the gum above its melting point during the addition of Part A ingredients. Store the gel in sealed container away from heat and air.

Formulation 18
Berri-Seal: A Natural Emollient Topical Delivery System

| | Ingredient | Function(s) | Wt. % |
|---|---|---|---|
| A | Poly(dimethylsiloxane) | Skin protectant | 0.8 |
| | DC-200 Fluid ® | | |
| | Decamethylpentane | Skin protectant | |
| | Cyclosiloxanes | | 0.33 |
| | DC-245 Fluid ® | | |
| | Mineral Oil | Oleophilic phase | 4.1 |
| | Berry Wax | Oleophilic phase | 9.3 |
| | EnviroPure310 ® | | |
| B | Deionized Water | Water phase (hydration) | 72.7 |
| | Corn Starch | Thickener (gel/ | |
| | | film-forming agent) | 3.1 |
| | Glycerol | Humectant | 10.0 |
| C. | CITRICIDAL | Natural preservative | 0.5 |

### Procedure

Weigh the Part B starch ingredient and place in suitable vessel equipped with mixer. Add a sufficient volume of deionized water, glycerol, mix thoroughly, and heat the Part B ingredients at 90° C. until the starch is entirely dissolved. Remove from heat and cool to 65° C. Weight out Part A ingredient (mineral oil, DC-200, DC-245 and Berry Wax/Olive Oil, EnviroPure310, React-NTI) and add directly to pre-heated Part B ingredients. Stir in Part C ingredient, and mix continuously until a homogeneous emulsion is formed. Keep the gelated starch above its boiling point during the addition of Part A ingredients. Store the gel in sealed container away from heat and air. A variety of pure food grade cornstarches can be employed including Stanley, National Starch, and Argo.

There has thus been shown and described a novel thixotropic miroemulsion which may be used as skin treatment, and a topical delivery system for plant derived anti-irritants, which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

1. A composition comprising athixotropic microemulsion comprising from about 1-4% starch, water, an emulsifier, and from about 1 to about 20% oil, said starch selected from the group consisting of natural starches, hydrophobic starches, chemically modified starches, and polysaccharides; said composition comprising a stable oil-in-water emulsion.

2. The composition of claim 1, wherein the average particle size distribution of the microemulsion is clustered around about 0.5 to 3 microns.

3. The composition of claim 1, wherein the average particle size distribution of the microemulsion is clustered around about 0.3 to 5 microns.

4. The composition of claim 1, wherein the average particle size of the microemulsion is 0.8 microns.

5. The composition of claim 1, wherein the starch is selected from the group consisting of corn starch and potato starch.

6. The composition of claim 1, wherein the emulsifier is a surfactant, selected from the group consisting of anionic surfactant, sodium lauryl sulfate, or a cationic surfactant such as benzalkonium chloride, or a non-ionic surfactant such as Triton X-100.

7. The composition of claim 1, wherein the emulsifier is sodium lauryl sulfate, said composition having a starch:oil ratio of from about 1:3 to 3:1

8. The composition of claim 1, further comprising a humectant, selected from the group consisting of glycerol, Trehalose, and sugar esters.

9 The composition of claim 1, wherein the oil comprises silicone type oils.

10. The composition of claim 9, wherein the silicon oils comprise dimethicon and decamethylcyclopentasiloxane.

11. The composition of claim 1, wherein the oil is selected from the group consisting of mineral oil, berry wax, petrolatum jelly and silicone oils.

12. The composition of claim 1, wherein the oil is selected from the group consisting of paraffin oil, polydimethylsiicone oil, perflurodecalin, and vegetable oils.

13. The composition of claim 1, further comprising an active agent.

14. The composition of claim 1, wherein the oxygen carrier is perflurodecalin.

15. The composition of claim 14, wherein the preservative is Citricidal.

16. The composition of claim 14, wherein the preservative is Tea Tree Oil.

17. The composition of claim 1, further comprising a natural preservative.

18. A composition comprising a thixotropic microemulsion comprising from about 1-4% starch, water, an emulsifier, and from about 1-20% oil, said starch selected from the group consisting of natural starches, hydrophobic starches, chemically modified starches, and polysaccharides; said composition comprising a stable oil-in-water emulsion, and having a molecular weight greater than about 300,000 daltons.

19. A topical gel drug delivery system comprising the thixotropic microemulsion of claim 1, and an anti-irritant selected from the group consisting of hydrophobic plant extracts and hydroalcoholic plant extracts.

20. A topical gel drug delivery system of claim 19, wherein the anti-irritant is Bisabol.

21. A topical gel drug delivery system of claim 19, wherein the preferred plant extracts are selected from the group consisting of Autumn Olive berries, corn tassel, grapefruit seed oil, Sea Buckthorn oil, green onion leaves, red swiss chard, red seedless grapes green tea leaves, hops (Humulus Lupulus), dired catkins from Linden trees (Tilia sp.), dried catkins of Oak Trees (Quercus, sp.), dried flowers of Lavender (Lavendar), dried cocoa powder, and dried cinnamon powder.

22. A topical gel drug delivery system of claim 19, wherein the preferred plant extracts are fruit extracts.

23. A topical gel drug delivery system of claim 19, further comprising AHA's.

24. A topical gel drug delivery system of claim 19, further comprising retinol

25. A topical gel drug delivery system of claim 19, further comprising Palmitoleic Acid

26. A method of making a stable oil-in-water emulsion, comprising:
A) mixing a starch in cold water to form a slurry, and adding a minor amount of an emulsifier,
B) heating and stirring the mixture until the starch undergoes thermal melt, and the mixture is clarified,
C) ceasing to heat the clarified mixture, and cooling it to a temperature just above the melting point of the oil to be emulsified, and
D) using low shear mixing, adding oil to the clarified mixture to form a thixotropic microemulsion, comprising from about 1-4% starch and from about 1 to about 20 % oil.

27. The method of claim 26, wherein the starch is selected from the group consisting of natural starches, hydrophobic starches, chemically modified starches, and polysaccharides.

28. The method of claim 26, further comprising the step of adding fragrance to the thixotropic microemulsion.

29. The method of claim 26, further comprising the step of adding Citricidal to the thixotropic microemulsion.

30. The method of claim 26, wherein the oil is mineral oil, and the Citricidal is added to the mineral oil in step A.

31. The method of claim 26, wherein Palmitoleic acid, in alcohol, is added to the mixture in step A.

32. The method of claim 26, further comprising the step of adding vegetable coloring to the cold water in Step A.

33. An alcohol resistant moisturizing, flexible skin layer comprising the thixotropic microemulsion of claim 1.

34. A moisturizing sanitary glove, comprising a skin layer comprising the thixotropic microemulsion of claim 1.

35. A wound dressing comprising a skin layer comprising the thixotropic microemulsion of claim 1.

36. A stomal dressing comprising a skin layer comprising the thixotropic microemulsion of claim 1.

37. An anti-bacterial wound dressing comprising the composition of claim 1, further comprising Palmitoleic acid.
